(19) United States
(12) Patent Application Publication
(10) Pub. No.: US 2021/0093454 A1
(43) Pub. Date: Apr. 1, 2021

(54) SYSTEMS AND METHODS FOR RESHAPING A HEART VENTRICLE

(71) Applicant: Ancora Heart, Inc., Santa Clara, CA (US)

(72) Inventors: Russel SAMPSON, Palo Alto, CA (US); Jeffrey M. CLOSS, Menlo Park, CA (US)

(21) Appl. No.: 17/033,283

(22) Filed: Sep. 25, 2020

Related U.S. Application Data
(60) Provisional application No. 62/906,524, filed on Sep. 26, 2019.

(51) Int. Cl. A61F 2/24

(52) U.S. Cl. CPC A61F 2/2487; A61F 2250/001; A61F 2220/0075; A61F 2220/0016

(57) ABSTRACT

Described herein are devices and methods for reshaping a heart ventricle. In one variation, a method may include securing an implantable, cinchable device to a ventricle wall, cinching the device by tensioning the tether until a circumferential portion of the ventricle at a location of the device is reduced by approximately 30% (e.g., from about 25% to about 35%), and locking the device in a cinched configuration. In one variation, the cinchable device has a plurality of tethered anchors and force distributing members. The cinchable device may be secured to the ventricle at a location approximately 10-20 mm below the mitral valve in a plane substantially parallel to the mitral valve, such that it spans approximately 220-230 degrees of the circumference of the ventricle at the location of the device. Some variations further comprise introducing a pre-determined amount of slack into the device before the device is lock in a tensioned state.

### CROSS-REFERENCE TO RELATED APPLICATIONS

This application claims priority to U.S. Provisional Patent Application No. 62/906,524, filed on Sep. 26, 2019, which is hereby incorporated by reference in its entirety.

### TECHNICAL FIELD

The systems and methods described herein relate generally to reshaping (e.g., reverse-remodeling) a heart ventricle.

### BACKGROUND

Many surgical therapies for functional mitral valve regurgitation (FMR) have been developed that treat the mitral valve annulus. Examples include the Carpentier ring annuloplasty and Kay annuloplasty procedures, which achieve annular reduction, as well as the Alfieri stitch, which coapts the MV leaflets using a suture. Percutaneous procedures have also been developed that adapt these surgical procedures to catheter-based approaches. These therapies aim to treat the symptoms of the underlying cardiomyopathy (e.g., mitral valve regurgitation) by modifying cardiac tissue in the vicinity of the mitral valve, for example, from the atrial side of the mitral valve and/or the ventricular side of the mitral valve (e.g., the mitral valve annulus).

Although these devices and methods may have some degree of success in addressing mitral valve regurgitation, they often do not address the underlying causes of cardiomyopathy, such as pathological remodeling of the ventricle. Accordingly, improved devices and methods are desirable.

### SUMMARY

Disclosed herein are methods for reshaping (e.g., reverse-remodeling) a heart ventricle using an implantable device. Generally, a method of reshaping a heart ventricle may comprise securing an implantable device into ventricular wall tissue below a mitral valve plane such that is spans along more than half of a circumference of the heart ventricle. A method for reshaping a heart ventricle may comprise securing an implantable device approximately 10-20 mm (e.g., about 10-15 mm) below a mitral valve plane across approximately 220-230 degrees of a circumference of the heart ventricle. Some methods may comprise securing the implantable device into ventricular wall tissue that is from about 3 mm to about 25 mm below a mitral valve plane (e.g., from about 7 mm to about 20 mm, from about 10 mm to about 15 mm), for example, securing the implantable device in myocardium bounded by the mitral valve plane (and/or the subannular groove) and papillary muscle insertion. The ventricular wall tissue where the implantable device is secured may be located between the mitral valve plane and a papillary muscle insertion location. In some variations, the device may comprise a plurality of anchors coupled to a tether and implanting the device may comprise implanting the plurality of anchors into the ventricle. The method may also comprise cinching the implantable device from an uncinched configuration to a cinched configuration such that a circumferential portion of the ventricle at the location of the device is reduced by approximately 30% (e.g., from about 25% to about 35%). In some variations, cinching the device may comprise tensioning a tether. The method may further comprise locking the implantable device in the cinched configuration. Locking the implantable device in a cinched configuration may comprise securing a lock member to the terminal end of the implantable device. In some variations, locking the implantable device in the cinched configuration may further comprise introducing a pre-selected amount of slack into the tether. In some variations, the plurality of anchors may comprise a first anchor and a terminal anchor. Introducing a pre-selected amount of slack to the tether may comprise securing a lock member on the tether at a pre-selected distance from the terminal anchor when the implantable device is in the cinched configuration.

In some variations of the methods described herein, the implantable device may extend along a circumference of the ventricle between a junction of a septum and a ventricular free wall adjacent to the mitral valve P3 leaflet, and a ventricle outflow tract. In some variations, the implantable device may have an uncinched length (i.e., when it is in an uncinched configuration) such that a ratio R between the uncinched length and the inner diameter of the ventricle at the end of diastole has a magnitude of at least two. Securing the implantable device may comprise securing a total of 11-16 anchors into the ventricle wall tissue. Further, securing the implantable device may comprise securing each anchor to the ventricle wall approximately 10-15 mm inferior to the mitral valve. In some variations, securing the implantable device into the ventricle wall tissue comprises deploying the plurality of anchors into the ventricle wall simultaneously.

Some variations of an implantable device may comprise a plurality of force distributing members, where each force distributing member is coupled to the tether between two anchors. Furthermore, securing the implantable device to the ventricle wall may comprise positioning a multi-window delivery catheter in the ventricle approximately 10-15 mm inferior to the mitral valve annulus. In some variations, the multi-window catheter may comprise a reinforced distal end comprising a pre-defined curvature that approximates a curvature at the widest circumference of the ventricle.

### BRIEF DESCRIPTION OF THE DRAWINGS

FIG. 1 depicts a flowchart representation of one variation of a method for reshaping (e.g., reverse-remodeling) a ventricle of a heart.

FIG. 2 depicts a variation of a device for reshaping a ventricle of a heart.

FIG. 3 depicts a variation of a tissue anchor.

FIG. 4A depicts one variation of a device for positioning a device within a ventricle.

FIG. 4B depicts one variation of a device for positioning a device within a ventricle.

FIG. 4C depicts one variation of a device for positioning anchors within a ventricle.

FIG. 5A depicts a perspective view of a distal portion of a device for positioning anchors in a ventricle.

FIG. 5B depicts a cross-sectional view of a device for positioning anchors at a location in the ventricle.

FIG. 6 depicts a schematic representation of a device for aligning an implant in a ventricle.

FIG. 7A depicts a schematic representation of a device for reshaping a ventricle implanted within a ventricle of a heart.

FIG. 7B depicts a schematic view of a mitral valve from the left atrium.

FIG. 8A depicts a schematic representation of one variation of a system for implanting a device into a ventricle.

FIG. 8B depicts a variation of an anchor delivery catheter.

FIG. 8C depicts a side view of a distal portion of the anchor delivery device of FIG. 8B.

FIG. 8D depicts a schematic representation of one variation of a system for implanting a device into a ventricle.

FIG. 9A depicts a schematic representation of a device implanted in a ventricle.

FIG. 9B depicts a schematic representation of a ventricle wall.

FIG. 10A depicts one variation of a lock member.

FIG. 10B depicts one variation of a lock catheter.

FIG. 10C depicts a side view of a distal portion of a lock catheter.

FIG. 11 depicts a schematic representation of a device implanted in a ventricle.

FIG. 12A depicts a fluoroscopic image of one example of a device implanted in a ventricle.

FIG. 12B depicts a fluoroscopic image of one example of a device implanted in a ventricle.

FIG. 12C depicts a fluoroscopic image of one example of a device implanted in a ventricle.

FIG. 12D depicts a fluoroscopic image of one example of a device implanted in a ventricle.

FIG. 13A depicts a fluoroscopic image of one example of a device implanted in a ventricle.

FIG. 13B depicts a fluoroscopic image of one example of a device implanted in a ventricle.

FIG. 13C depicts a fluoroscopic image of one example of a device implanted in a ventricle.

FIG. 13D depicts a fluoroscopic image of one example of a device implanted in a ventricle.

FIG. 14 depicts a table showing changes in ejection fraction percentage (EF %), left ventricle end diastolic volume (LVEDV), and left ventricle end systolic volume (LVESV) for seven patients over time using the devices and methods described herein.

FIG. 15A depicts a data plot that represents average patient ejection fraction (EF %) changes over time using different variations of implants and methods.

FIG. 15B depicts a data plot that represents individual patient ejection fraction (EF %) changes over time using other devices and methods, where each line represents the EF % data for an individual patient.

FIGS. 16A-16C depict bar graphs of average patient ejection fraction data (EF %) over multiple time intervals using the devices and methods described herein.

FIGS. 17A-C depict bar graphs of individual patient ejection fraction percentage (EF %) data over multiple time intervals using alternate methods and implants.

FIGS. 18A-18C depict bar graphs of average patient left ventricle end systolic volume data (LVESV) over multiple time intervals using the devices and methods described herein.

FIG. 18D depicts a table showing changes in ejection fraction percentage (EF %), and the end systolic volume index (ESVi) for patients treated using other mitral valve repair or replacement devices.

FIGS. 19A-19C depict bar graphs of average patient left ventricle end systolic diameter data (LVESD) over multiple time intervals using the devices and methods described herein.

FIGS. 20A-20C depict bar graphs of average patient left ventricle end diastolic volume data (LVEDV) over multiple time intervals using the devices and methods described herein.

FIGS. 21A-21C depict a series of data charts showing individual left ventricle end systolic volume (LVESV) and left ventricle end diastolic volume (LVEDV) over time using alternate devices and methods.

FIGS. 22A-22C depict bar graphs of New York Heart Association Classification (NYHA Class) data for patients over multiple time intervals using the devices and methods described herein.

FIGS. 23A-23C depict bar graphs of individual patient New York Heart Association (NYHA) Classification data over multiple time intervals using alternate devices and methods.

### DETAILED DESCRIPTION

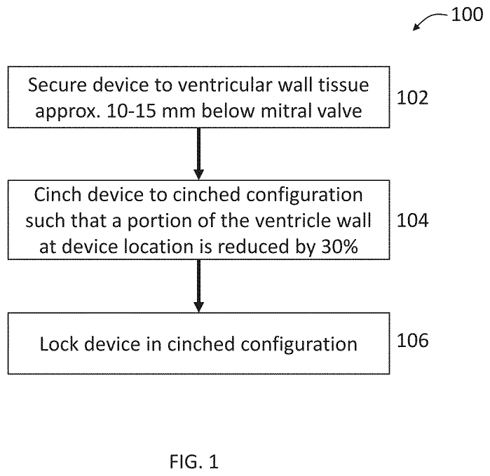

Described herein are exemplary variations of methods for reshaping (e.g., reverse-remodeling) a heart ventricle. FIG. 1 is a flowchart depicting one variation of a method 100 for reshaping a heart ventricle. The method 100 may comprise securing 102 an implantable device to a ventricular wall tissue approximately 10-15 mm below the mitral valve of a heart, cinching 104 the implantable device from an uncinched configuration to a cinched configuration by tensioning the tether until a circumferential portion of the ventricle wall at a location of the device is reduced by approximately 30% (e.g., from about 25% to about 35%), and locking 106 the implantable device in a cinched configuration. Some methods may comprise securing the implantable device into ventricular wall tissue that is from about 3 mm to about 25 mm below a mitral valve plane (e.g., from about 7 mm to about 20 mm, from about 10 mm to about 15 mm), for example, securing the implantable device in myocardium bounded by the mitral valve plane (and/or the subannular groove) and papillary muscle insertion. In some variations, the implantable device may comprise a plurality of anchors coupled to a tether. Locking the implantable device in a cinched configuration may further comprise cinching the implant to its hard stop, then introducing a pre-determined amount of slack back into the implantable device by securing a lock member to the tether at a pre-defined distance from the terminal anchor of the device.

Although one or more aspects of the methods described herein appear to be contrary to common practices and concepts in heart failure management, cardiac valve repair, and/or LV and cardiac function improvement, the combination and/or synergy of these aspects unexpectedly results in the enhancement of therapeutic ventricular reshaping (e.g., reverse-remodeling). Enhanced therapeutic ventricular reshaping (e.g., reverse-remodeling) may help to improve heart failure symptoms, decrease the severity of mitral valve regurgitation and improve overall cardiac function. For example, heart failure symptom management usually consists of guideline directed medical therapy (GDMT) followed by cardiac resynchronization therapy (CRT) if indicated. Device-based interventional techniques are not typically used in the treatment of heart failure unless the patient's symptoms deteriorate to the point of being placed on a left ventricular assist device (LVAD) and transplant list.

Although device-based techniques are not typically used for addressing heart failure, they are often used for mitral regurgitation. However, it has been a long-standing practice and widely-accepted teaching that to effectively address mitral valve regurgitation, an implant should be secured at or near the mitral valve annulus, which may directly modify the geometry of the mitral valve from either the atrial side or the ventricular side. In contrast, the methods described herein instead comprise securing the implantable device to the ventricular wall tissue bounded by the mitral valve plane (and/or subannular groove) and the papillary muscle insertion (e.g., from about 3 mm to about 25 mm below the mitral valve, from about 7 mm to about 20 mm below the mitral valve, from about 10 mm to about 15 mm below the mitral valve). In addition, it was previously-understood that an effective approach to reducing mitral valve regurgitation and preventing blood leakage backward into the atrium would be to bring the leaflets closer together by, for example, drawing the edges of the valve annulus and/or leaflets together as tightly as possible. In contrast, the methods described herein comprise reducing a circumference of a ventricle (e.g., at the location of the device) by about 30% (e.g., from about 25% to about 35%) and/or including a degree of flexibility in the cinchable device such that the device does not overly-restrict or hinder the motion of the cardiac walls as it beats. Without wishing to be bound by theory, it is believed maintaining a degree of flexibility in the cinchable device may facilitate ventricular movement during systole. Restricting cardiac wall motion by over-tightening the ventricle circumference may encourage pathological cardiac remodeling. Furthermore, the amount of cinching to reshape the mitral valve when the device is secured at or near the mitral valve annulus is not the same as the amount of cinching to affect ventricle reshaping when the device is secured into ventricular wall tissue at a location below the mitral valve annulus and above the papillary muscle insertion. In some variations where the implantable device comprises a plurality of tethered anchors, including a degree of flexibility may comprise introducing a pre-determined amount of slack before the device is locked in its cinched configuration so that the ventricular walls have a greater range of motion as the heart contracts during systole and expands during diastole.

In some variations, the implantable device may comprise a tether and a plurality of anchors coupled to the tether. The implantable device may further comprise a plurality of force distribution members (FDMs) disposed over the tether and located between the anchors. The implantable device may be secured to the ventricle in any suitable manner. For example, securing the implantable device may comprise implanting a plurality of anchors coupled to a tether to a predetermined location in the ventricle. Anchors may be configured to pierce through the ventricular wall tissue in order to secure the implantable device to the ventricle. Anchors may also be configured to maintain the position of the implantable device in the ventricle after implantation to facilitate therapeutic cardiac remodeling. Securing the implantable device may comprise positioning a multi-window catheter in the ventricle at a desired implantable device location. For example, securing the implantable device may comprise positioning a multi-window catheter at a location approximately 10-15 mm below the mitral valve in a plane, and may optionally be substantially parallel to a plane of the mitral valve annulus. Some methods may comprise securing the implantable device into ventricular wall tissue that is from about 3 mm to about 25 mm below a mitral valve plane (e.g., from about 7 mm to about 20 mm, from about 10 mm to about 15 mm), for example, securing the implantable device in myocardium bounded by the mitral valve plane (and/or the subannular groove) and papillary muscle insertion. Securing the implantable device may further comprise extending a first anchor delivery catheter through a lumen of the multi-window catheter. Securing the implantable device may also comprise advancing the anchor delivery catheter out of an opening of the multi-window catheter to a target anchor location in the ventricle wall. Securing the implantable device may then comprise deploying an anchor into tissue at the target anchor location. Securing the implantable device may further comprise delivering a plurality of anchors via the multi-window catheter using a plurality of anchor delivery catheters.

It should be understood that although the examples described herein include an implantable device comprising a plurality of anchors and force distribution members coupled to a tether, a method for reshaping (e.g., reverse-remodeling) a ventricle may comprise any suitable device that is configured to cinch, tighten, compress, and/or reduce a dimension of the ventricle and/or valve. Any device that is configured to cinch, tighten, shrink, tension, plicate, and/or otherwise draw tissue together may be used with any of the methods described herein. For example, one variation of an implantable device may comprise a flexible sheath coupled to a plurality of springs configured to penetrate and attach to the ventricle wall. The sheath may further comprise a wire or thread “spine” threaded through the sleeve such that tensioning the spine cinches the device. Other examples may comprise a flexible sleeve that may be attached to the ventricle wall using sutures, or a conformable shape memory device.

### Implantable Device Components

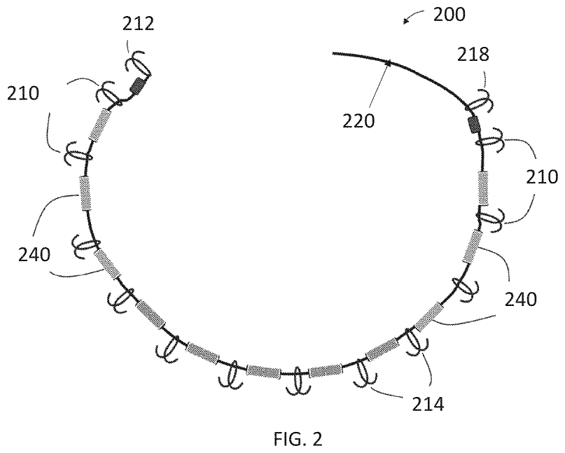

FIG. 2 depicts one variation of an implantable device that may be secured to a ventricular wall for reshaping a heart ventricle (e.g., reverse-remodeling the ventricle in order to reverse the effect of pathological cardiac remodeling). An implantable device 200 may comprise a plurality of tethered anchors 210. The implantable device 200 may comprise a first distal-most anchor 212, one or more secondary anchors 214, and proximal-most terminal anchor 218. The first anchor 212 may be fixedly attached (e.g., knotted, adhered, welded, etc.) to the tether 220. The plurality of secondary anchors 214 may be slidably coupled to the tether 220. For example, the tether may be threaded through an opening in each secondary anchor 214. The terminal anchor 218 may also be slidably disposed about the tether 220. In another variation the terminal anchor 218 may also be fixedly attached to the tether 220. For example, the tether may be fixedly attached (e.g., knotted, adhered, welded, etc.) to the proximal-most terminal anchor 218. Each anchor 210 may be configured to attach to a portion of the ventricle wall. For example, a portion of each anchor may pierce through and penetrate the tissue of the ventricle wall to secure the implantable device 200 to the ventricle. Force distribution members (FDMs) 240 may be disposed about the tether 220 between or adjacent to all or a subset of the anchors 210. For example, one FDM 240 may be located between each set of two sequential anchors 210. The implantable device may further comprise a lock member, which may operate to secure the implantable device in a cinched configuration, as discussed below.

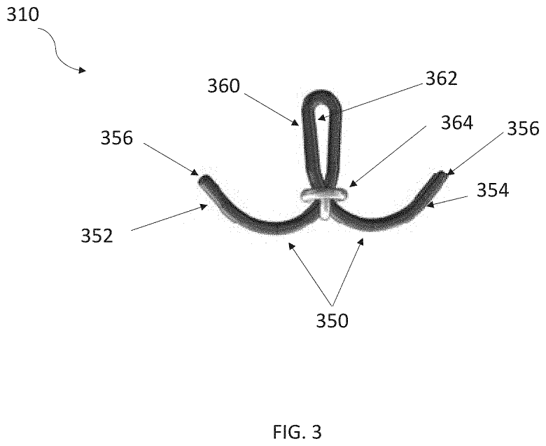

In some variations, an implantable device may comprise a plurality of tethered tissue anchors. The anchors may comprise a tissue-attachment structure and a tether-coupling structure. FIG. 3 depicts one variation of an anchor 310 of an implantable device that may be configured to be secured into the ventricle wall. The anchor 310 may comprise a tissue attachment portion 350 and an eyelet or loop portion 360 that is configured to retain a tether. The tissue attachment portion 350 may be configured to secure the anchor 310 to the ventricle wall, and the eyelet portion 360 may comprise an opening configured to house a tether. For example, as depicted in FIG. 3, the tissue attachment portion 350 may comprise a first leg 352 and a second leg 354, each leg having a tissue-piercing end 356 for penetrating cardiac tissue (e.g., piercing through the surface of myocardium), and one or more curves along the length of each leg to engage cardiac tissue. The eyelet portion 360 may be a loop with a central opening 362, such that the tether may be threaded through the opening 362. The anchor may be made of a single, continuous wire (e.g., of nitinol) that extends in a single-turning direction from one end to the other end (e.g., ends 356), forming a loop (e.g., eyelet 362) in between the ends. In other variations, an anchor may comprise multiple components secured together, such as by welding, adhesive, or any other suitable methods. For example, the eyelet portion and tissue attachment portion may be comprised of two or more separate wire segments that are secured together. Optionally, the anchor 310 may also comprise a ring-shaped wire, suture or collar 364 that is located at the base of the eyelet or loop 360. The collar 364 creates a closed loop in the eyelet to prevent the anchor from detaching from the tether, and may help to secure the eyelet and/or reinforce the size and shape of the eyelet portion 360. Other anchor variations may not have a collar. The eyelet or loop 360 may have any suitable shape. For example, the eyelet portion 360 may have an elongate shape and/or a narrow profile that tapers to the base, which may facilitate tissue penetration. Alternatively, some anchors may not comprise two legs and an eyelet between them. For example, an anchor may comprise one hook and one loop, having an S-shaped configuration. Alternatively or additionally, an anchor may comprise a plurality of struts attached to a loop. Devices described herein may comprise any suitable tissue attachment devices such as clips, clamps, springs, hooks, sutures, etc.

The anchor may be comprised of any conformable and/or elastic material. For example, the anchor (either or both the tissue attachment portion and the eyelet or loop portion) may be made of an elastic material (e.g., a super-elastic material) and/or a shape-memory material. Examples of such materials may include any metals, alloys, such as nickel titanium alloy (Nitinol), or polymers (e.g., rubber, poly-ether ether ketone (PEEK), polyester, nylon, etc.). The anchor may also comprise more than one material. For example, in some variations, the tissue attachment/penetration portion and eyelet portion of the anchor may be comprised of nitinol, and the collar of the anchor may be comprised of polyester. In some variations, the anchor or the collar or both may comprise a radiopaque material. This may provide visibility of the anchors while they are being secured to the ventricle, which may aid the implantation of the device. In some variations, portions of the FDMs may comprise a radiopaque material. Using one or more radiopaque materials in the anchors and/or FDMs may permit fluoroscopic images of the devices to be acquired during implantation, which may facilitate the placement of the implant at the desired locations and with the desired orientation.

Turning back to FIG. 2, the implantable device 200 may further comprise one or more force distribution members (FDMs) 240 slidably coupled to the tether and situated between the plurality of anchors 210, as depicted in FIG. 2. Each FDM 240 may be situated between two anchors 210. In some variations of the implantable device, a FDM 240 may be situated adjacent and distal to the first anchor 212. Similarly, a FDM may be situated adjacent to and proximal to the terminal anchor 218. Any suitable number of FDMs may be positioned between or adjacent to anchors. For example, in some variations, two or more FDMs may be positioned between two anchors. Different numbers of FDMs may be used at different positions of the device. For example, two FDMs may be positioned between the terminal anchors and next-to-terminal anchors (e.g., between the distal-most terminal anchor and the next-to-distal-most anchor, and/or between the proximal-most terminal anchor and a next-to-proximal-most anchor), while one FDM may be positioned between all other sets of anchors. In another variation, one FDM may be positioned between the terminal anchors and next-to-terminal anchors, while two FDMs may be positioned between all other sets of anchors. It is not necessary that the distribution of FDMs be uniform, and any number of FDMs may be positioned adjacent to any anchor. For example, one FDM may be positioned between the proximal-most terminal anchor and a next-to-proximal-most anchor, and subsequent FDMs may be positioned between every-other set of anchors. In one variation, the FDMs 240 may all be the same length. However, in another variation, the FDMs may have varying length. For example, the FDMs near the center of the implantable device may be longer or shorter in length than the FDMs of at the ends of the implantable device. In one variation, the FDMs between the terminal anchors and the next-to-terminal anchors (e.g., between the distal-most terminal anchor and the next-to-distal-most anchor, and/or between the proximal-most terminal anchor and a next-to-proximal-most anchor) may be shorter than the FDMs between the intermediate anchors (e.g., the anchors in a central region of the implant). However, the FDMs may be of any suitable length.

Further, the FDMs 240 may have a lumen configured to house the tether 220. Thus, the FDM may be slidably disposed about the tether by extending the tether though the lumen of the FDM. In the variation depicted in FIG. 2, the FDMs 240 have a tubular configuration. However, the FDMs 240 may be of any suitable shape. For example, the FDMs may comprise a rectangular, ovular, or triangular cross section. FDMs may be comprised of a single component, or a series of components, for example a series of spherical components (e.g., ovoid, elliptical, or spherically-shaped beads or “pearls”). FDMs may be of any suitable material. For example, the FDMs may be made of nitinol, polymer, plastic, polyester, or metal. Further, the surface of the FDM may be textured and/or be coated. For example, the surface of the FDM may have a pattern of cutouts and/or ridges, which may help facilitate integration with cardiac tissue. Optionally, a FDM may comprise a coating or fabric that may help induce tissue formation and incorporation such that shortly after implantation, the implant may become at least partially incorporated into the wall of the left ventricle (LV). Further, one or more portions of a FDM may comprise a radiopaque material, such as barium sulfate. The radiopaque material may be distributed throughout the FDM 240 and/or may be concentrated at particular regions or bands on the FDM, as may be desirable. This may provide visibility of the force distribution members while they are being implanted, which may aid the implantation of the implantable device at the desired location and/or orientation (e.g., as specified in FIG. 1). It may be beneficial for a user to be able to see the force distribution members while the implantable device is being implanted so that the user can see the location of the implantable device within the ventricle, and ensure the implantable device is properly situated, for example. A FDM may be made entirely of bioabsorbable or biodegradable materials, entirely of non-bioabsorbable or non-biodegradable materials, or may be a composite structure where some portions are bioabsorbable or biodegradable and some portions are not. Other variations of force distribution members are discussed in U.S. Pat. Appln. Pub. No. 2018/0140421, which is hereby incorporated by reference in its entirety.

In some variations, methods of reshaping a heart ventricle may comprise implanting a device into ventricular wall tissue approximately 10-15 mm below a mitral valve plane, and cinching the device from an uncinched configuration to a cinched configuration such that a circumferential portion of the ventricle at a location of the device is reduced by approximately 30% (e.g., from about 25% to about 35%). Alternatively or additionally, methods may comprise securing the implantable device into ventricular wall tissue that is from about 3 mm to about 25 mm below a mitral valve plane (e.g., from about 7 mm to about 20 mm, from about 10 mm to about 15 mm), for example, securing the implantable device in myocardium bounded by the mitral valve plane (and/or the subannular groove) and papillary muscle insertion. Methods may further comprise securing the device in a cinched configuration. The implantable device need not comprise a plurality of anchors coupled to a tether and may be any device that is configured to cinch, tighten, shrink, tension, plicate, and/or otherwise draw tissue together. One variation of an implantable device that may be used with any of the methods described herein may comprise a sleeve or sheath coupled to one or more helical tissue-penetrating members which may be secured into the ventricular wall of a heart. Helical tissue-penetrating members may be, for example, prongs or screws having external helical threads, helical fasteners (e.g., corkscrew-like fasteners) having a pointed tissue-piercing tip, and the like. The sheath may be comprised of flexible material. The sheath may further comprise a wire or thread “spine” threaded through the sleeve such that tensioning the spine cinches the device. To affix the sheath or sleeve to the ventricular wall, the helical tissue-penetrating members may be located within or partially within the sleeve or sheath such that the tissue-penetrating members penetrate through the sleeve or sheath into the ventricular wall tissue, or the helical tissue-penetrating members may be fixedly attached to a surface of the sheath. A knot and/or lock member may be secured to the spine in order to maintain the device in a cinched configuration. The spine may be tensioned in any suitable manner. For example, a catheter may be configured to rotate the lock member to tension the spine. The device may comprise one or more radiopaque components to aid in visualization during implantation. In another variation, an implantable device may comprise a flexible sleeve and one or more sutures coupled to the sleeve. The sutures may be slidably threaded through the sleeve, and through the ventricular wall to attach the sleeve to the ventricle. Tensioning a suture that has been threaded throughout the sleeve and into and out of ventricular wall tissue may cinch the implant and the ventricular wall tissue. Some variations of implantable devices may be self-cinching. For example, an implantable device may be made of shape-memory material such that the device can be implanted in a restrained, uncinched configuration, and revert to a cinched configuration when it is no longer restrained or under tension. For example, an implantable device may comprise a portion made of shape memory material coupled to attachment mechanisms such as anchors, sutures, or helical tissue penetrating members. The attachment mechanisms may secure the shape memory portion of the device to the ventricular wall. In some variations, the attachment mechanisms may be secured to the tissue while the device is in an uncinched configuration (e.g. under tension), and the device may be released from tension or unrestrained such that it reverts back to a cinched configuration, thereby applying force to reduce the circumference of the ventricular wall at the location of the device. For example, a shape memory device may be restrained in an uncinched configuration by a delivery catheter, and released from the catheter after implantation into ventricular tissue, allowing the device to revert to a cinched configuration. In another variation, an implantable device made of shape memory material may be placed under tension as it is implanted. Once fully implanted, the tendency of the device to revert back to an uncinched configuration may apply a cinching force to the ventricular wall. Such a device would not require a separate step to cinch the device, but would automatically assume the cinched configuration once unrestrained. Any of the device and methods described herein may be used to implant the device in the desired location and desired orientation, and cinch and lock the device by a desired amount. Alternatively, other methods and/or device may be used to implant, lock, and cinch an implantable device.

As described above, methods for reshaping (e.g., reverse-remodeling) a heart ventricle may comprise securing an implantable, cinchable device at a location about 10-15 mm below the mitral valve, and optionally in a plane approximately parallel to the plane of the mitral valve (e.g., a plane defined by the mitral valve annulus or a plane defined by one or more of the mitral valve leaflets). The device may also be implanted at an angle a1 to the mitral valve plane, and in some variations, may be longer (e.g., has more tissue anchors and FDMs, has a greater length between the distal anchor and proximal anchor) than a device that is implanted at an angle a2 to the mitral valve plane, where angle a2 is less than angle a1 (e.g., a device that is implanted approximately parallel to the mitral valve plane). Securing the implantable device 200 may comprise securing a plurality of tethered anchors into the ventricle wall. Securing an implantable device into the ventricle wall may comprise using various catheters to position and secure the plurality of anchors to a location within the ventricle. For example, securing an implantable device to the ventricle may comprise using catheters to position and secure one or more anchors at a location approximately 10-15 mm below the mitral valve. Some methods may comprise securing the implantable device into ventricular wall tissue that is from about 3 mm to about 25 mm below a mitral valve plane (e.g., from about 7 mm to about 20 mm, from about 10 mm to about 15 mm), for example, securing the implantable device in myocardium bounded by the mitral valve plane (and/or the subannular groove) and papillary muscle insertion. In some variations, a multi-window catheter may be configured to facilitate the delivery of the implantable device at the desired or pre-determined location below the mitral valve. A multi-window catheter may comprise a distal portion having a pre-defined curvature that approximates the radius of curvature of the ventricle approximately 10-15 mm below the mitral valve and/or that approximates the radius of curvature of the widest portion of the ventricle. Optionally, the distal portion of the multi-window catheter may be stiffened relative to its proximal portion so that the distal portion retains its curvature in a beating heart procedure, which may facilitate positioning or seating the multi-window catheter along the ventricular wall approximately 10-15 mm apical to the mitral valve. A stiffened distal portion that has a radius of curvature that approximates, or is larger than, the radius of curvature of the ventricle at or around the desired implantation location may help the multi-window catheter seat itself at or around the implantation location while the heart is beating. As described above, securing a cinchable implantable device approximately 10-15 mm below the mitral valve annulus may facilitate therapeutic remodeling of a heart ventricle as compared to methods that secure a cinchable implantable device at locations closer to the mitral valve (e.g., at the mitral valve annulus).

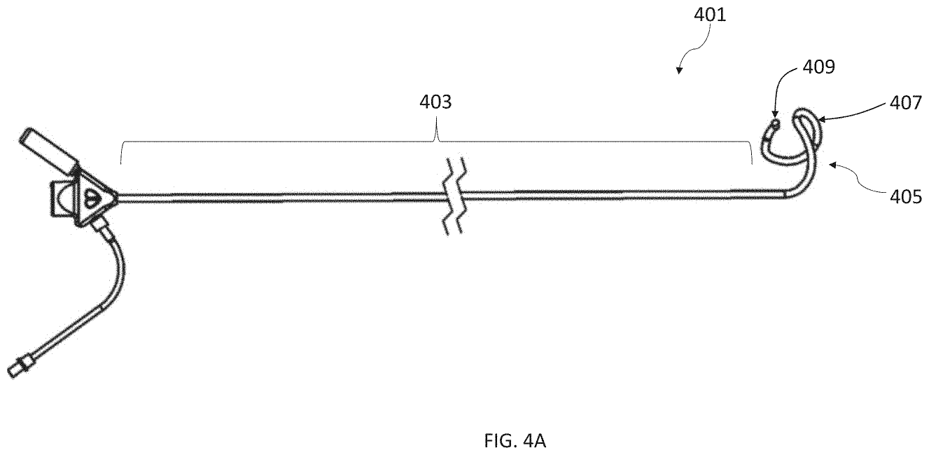

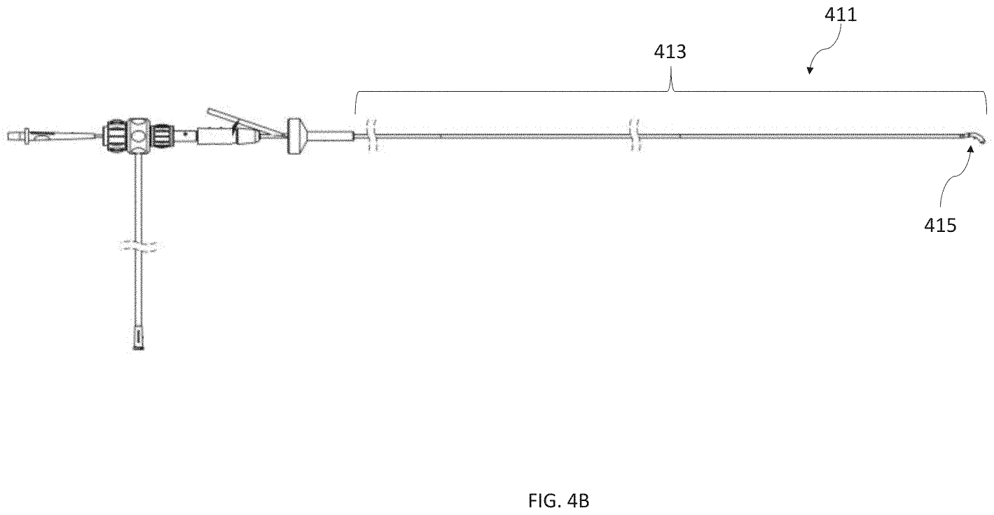

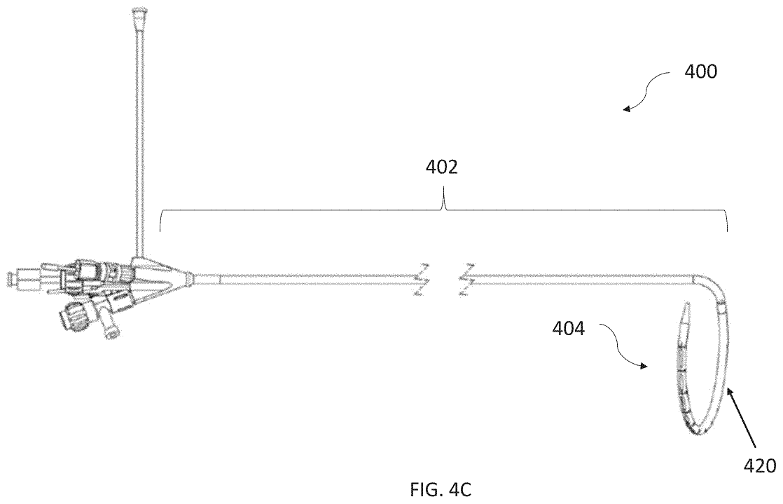

A method of securing an implantable device to the ventricle may comprise positioning a multi-window catheter in the ventricle to deliver the implantable device at a pre-selected location in the ventricle. For example, securing the implantable device to the ventricle may comprise advancing a guide catheter to ventricular tissue at or near the mitral valve (e.g., at or near the mitral valve annulus and/or subannular groove region in the left ventricle, along the anterolateral wall and in the sub-valvular space behind chordae tendineae) and advancing the multi-window catheter through the guide catheter, and positioning the multi-window catheter at a location below the mitral valve plane (e.g., from about 3 mm to about 25 mm below the mitral valve plane, from about 7 mm to about 20 mm below the mitral valve plane, from about 10 mm to about 15 mm below the mitral valve plane, in myocardium bounded by the mitral valve plane and papillary muscle insertion). FIG. 4A depicts one variation of a guide catheter 401 comprising an elongate body 403 and a distal portion 405 comprising one or more preshaped curves 407. The preshaped curves 407 may have contours and/or bends that correspond to the contours and/or bends of a patient's vasculature such that advancing and/or aligning the guide catheter along the contours and/or bends of the patient's vasculature automatically positions distal-most end 409 of the guide catheter at or near the mitral valve annulus and/or subannular groove region in the left ventricle. In some variations, the distal portion 405 of the guide catheter 401 may comprise a steerable, deflectable tip portion which may allow the curvature of the distal portion to be adjusted (e.g., by using a deflection knob on a proximal handle of the guide catheter). For example, the distal portion 405 may be adjusted such that the distal tip portion is located along the anterolateral wall and in the sub-valvular space behind chordae tendineae.

Optionally, a guidewire catheter may be advanced through the guide catheter to facilitate the placement of a guidewire along the ventricular wall regions where the device is to be implanted, and/or between the chordae tendineae and the endocardium. One variation of a guidewire catheter 411 is depicted in FIG. 4B. A guidewire catheter 411 may comprise an elongate body 413, a guidewire lumen in the elongate body 413, and a distal tip region 415. The distal tip region 415 may have one or more preshaped curves to facilitate positioning in the subannular groove (e.g., junction of left ventricular wall and mitral valve annulus) behind/around the chordae tendineae. The diameter of the elongate body 413 may be smaller than the diameter of the elongate body 403 of the guide catheter 401 so that the guidewire catheter 411 may be slidably advanced with a lumen of the guide catheter. In use, the guidewire catheter 411 may be advanced through the guide catheter 401 to the left ventricle, and a guidewire may be advanced through the guidewire catheter lumen to track around the circumference of the left ventricular wall. The guidewire catheter 411 may be advanced to track along at least a portion of the circumference of the left ventricular wall. After the position of the guidewire has been confirmed (e.g., using fluoroscopy or other suitable imaging methods), the guidewire catheter 411 may be withdrawn and the guidewire may be left in place to facilitate the positioning of other catheters and devices into the left ventricle. In some variations, the guidewire may be positioned along the subannular groove such that the distal tip of the guidewire exits the outflow tract and optionally re-crosses the aortic arch.

FIG. 4C depicts one variation of a multi-window catheter 400 that may be advanced through the guide catheter 401 (i.e., over the guidewire, after the guidewire catheter has been withdrawn). The multi-window catheter 400 may comprise an elongate body 402 with one or more pre-defined reinforced curvatures 420 at a distal portion or length 404 of the catheter. As described above, a pre-defined reinforced curvature 420 at a distal length 404 of the catheter 400 may facilitate placement of the distal length 404 of the catheter 400 at a desired location (e.g., approximately 3-25 mm below the mitral valve, approximately 10-15 mm below the mitral valve, approximately 7-20 mm below the mitral valve, between the mitral valve plane and papillary muscle insertion). FIGS. 5A and 5B depict one variation of a multi-window catheter 400 comprising an outer catheter 410 having a lumen 414 and a series of openings 412 in and along a sidewall 416 of the outer catheter 410. The placement of the openings along multi-window catheter may aid in securing the anchors in a desired configuration. For example, the spacing or distance between each opening may correspond to the desired spacing between anchors. In some variations, the spacing or distance between anchors (and the corresponding spacing or distance between each opening of the multi-window catheter) may be from about 6 mm to about 20 mm, e.g., from about 6 mm to about 12 mm, from about 8 mm to about 13 mm, from about 10 mm to about 15 mm, from about 15 mm to about 18 mm, from about 12 mm to about 20 mm, about 10 mm, about 11 mm, about 11.5 mm, about 12.5 mm, etc.). The number of openings 412 may correspond to the desired number of anchors to be implanted, though the number of anchors delivered can be greater (if more than one anchor is deployed from a window) or fewer than the number of openings. In some variations, a multi-window catheter may comprise an inner catheter that is slidable within the outer catheter. For example, FIG. 5B depicts a multi-window catheter comprising an outer catheter 410 and an inner catheter 450 slidable within the outer catheter 410. The inner catheter 450 may comprise a lumen and a sidewall opening, wherein aligning the side wall opening of the inner catheter with each of the sidewall openings of the multi-window catheter helps guide the sequential delivery of individual anchors through each of the sidewall openings to target anchor locations. Each of the anchors of the implantable device may be implanted into the ventricle wall at a pre-selected depth by deploying anchors through the openings in the inner and outer multi-window catheter when it is located at a pre-selected depth within the ventricle.

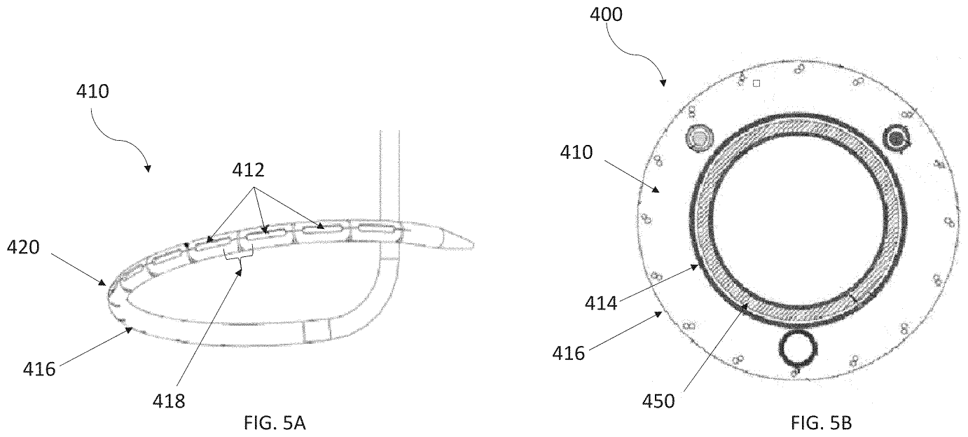

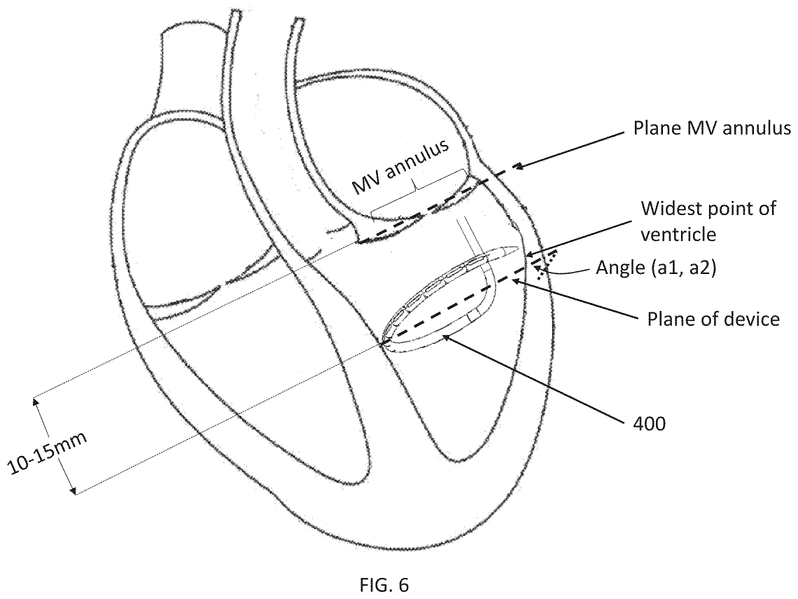

In some variations, the multi-window catheter may have a pre-defined reinforced curvature at a distal portion that approximates the curvature at the widest point of the ventricle to facilitate placement of the distal end of the catheter at a desired location. Alternatively or additionally, the pre-defined reinforced curvature may have a radius of curvature that approximates the circumference of the ventricle at a location about 10-15 mm below the mitral valve. As depicted in FIG. 6, a curvature approximating the curvature at the widest point of the ventricle may help to position the multi-window catheter 400 in the ventricle at a position approximately 10-15 mm below the mitral valve annulus. The radius of curvature of the distal portion of the multi-window catheter may be greater than radius of curvature of the ventricle. In some variations, the radius of curvature of the distal portion of the multi-window catheter may be from about 3 cm to about 6 cm (e.g., such that the diameter of the curvature of the multi-window catheter is from about 6 cm to about 12 cm). This may facilitate conformation of the ventricle and the multi-window catheter to each other, and may encourage consistent apposition between the multi-window catheter and the ventricular wall. This may provide the benefit of facilitating anchor delivery at a pre-determined distance of approximately 10-15 mm below the mitral valve annulus. As described above, the placement of the implantable device at this location may help facilitate therapeutic ventricular remodeling, alleviate mitral valve regurgitation, and facilitate cardiac function (e.g., improved ejection fraction, etc.). In some variations, the implantable device may be secured at any region in the ventricle between the mitral valve plane and the papillary muscle insertion location. Some methods may comprise securing the implantable device into ventricular wall tissue that is from about 3 mm to about 25 mm below a mitral valve plane (e.g., from about 7 mm to about 20 mm, from about 10 mm to about 15 mm), for example, securing the implantable device in myocardium bounded by the mitral valve plane (and/or the subannular groove) and papillary muscle insertion. The reinforced distal curvature may also provide the benefit of a stiffened profile that allows the reinforced curvature of the distal end of multi-window catheter to remain in a level plane once extended into the ventricle. This may facilitate the positioning of the multi-window catheter in a plane that is substantially parallel to the plane of the mitral valve annulus, as depicted in FIG. 6. It may be preferable to secure the implantable device to the ventricle wall in a plane that is substantially parallel to the plane of the mitral valve annulus, or at a known angle to the mitral valve plane. For example, a device may be implanted such that the first anchor and the last anchor are vertically displaced from each other, defining a device plane that is at a known angle to the mitral valve plane. Optionally, the length of a device implanted at an angle (a1, a2) relative to the mitral valve plane may be greater than the length of a device implanted parallel to the mitral valve plane. In one variation, two devices may be implanted opposite to each other, where the first device may be implanted at about +20 degrees to about +30 degrees to the mitral valve plane and the second device may be implanted at about at −20 degrees to about −30 degrees to the mitral valve plane.

The predefined reinforced curvature of the multi-window catheter may be reinforced by any suitable mechanism. For example, the distal end of the multi-window catheter may be composed of stiffer material than other portions of the catheter, and/or may comprise a wire or strengthened spine along the length of the distal end. Further, the curvature may be pre-defined in any suitable manner, such as by using a shape-memory material to form the distal curvature. In some variations, securing an implantable device to ventricular wall tissue may comprise visualizing the placement of the multi-window catheter before implanting the implantable device. Images and/or videos may be acquired to confirm that the multi-window catheter has been placed at the desired location in the ventricle (i.e. approximately 10-15 mm below the mitral valve) and in the desired orientation (i.e. approximately parallel to the plane of the mitral valve annulus). For example, the method may further comprise assessing the position of the multi-window catheter using fluoroscopy and injecting a contrast agent. In some variations, the multi-window catheter may comprise radiopaque components for enhanced visualization during a procedure.

As described above, methods herein may comprise delivering anchors to a pre-determined location in the ventricle. For example, it may be preferable to secure the anchors to a location approximately 10-15 mm below the mitral valve annulus. Some methods may comprise securing the implantable device into ventricular wall tissue that is from about 3 mm to about 25 mm below a mitral valve plane (e.g., from about 7 mm to about 20 mm, from about 10 mm to about 15 mm), for example, securing the implantable device in myocardium bounded by the mitral valve plane (and/or the subannular groove) and papillary muscle insertion. The multi-window catheter system may also facilitate repeatable delivery of anchors at target implant sites along the ventricular wall. For example, the plurality of openings of the outer catheter may permit delivery of tissue anchors with pre-determined spacing and/or alignment with respect to each other. The number and spacing of the openings of in the multi-window catheter may also dictate the span of the plurality of anchors across the ventricle wall. Turning to FIGS. 5A and 5B, the multi-window catheter 400 may comprise an outer catheter 410 comprising a plurality of openings 412 and a lumen 414, and an inner catheter 450 that is slidable within the lumen 414 of the outer catheter. The inner catheter may facilitate the placement of an anchor at a target anchor location by directing an anchor delivery catheter through a single side wall opening of the outer catheter while restricting or blocking access through the other side wall openings. Methods herein may comprise sliding the inner catheter 450 within the outer catheter 410 to reveal an unobstructed opening. For example, the inner catheter 450 may comprise an opening and may be slidable within the outer catheter. Thus, the inner catheter 450 can be aligned within the outer catheter 410 such that only one opening is left unobstructed. By aligning the inner catheter 450 with the outer catheter 410 in a particular fashion, only one opening will be available for the anchor delivery catheter to extend through and secure an anchor to a ventricle wall. Thus, the multi-window catheter system 400 may help reduce the likelihood that an anchor is deployed to an incorrect or undesired location. However, the inner catheter 450 may have any suitable number of openings. For example, it may be desirable to allow two openings in the sidewall of the outer catheter to remain unobstructed. Thus, the inner catheter may comprise two openings spaced apart to correspond with the openings in the outer catheter.

As described above, methods of securing an implantable device to the ventricle wall may comprise positioning anchors at pre-determined locations in the ventricle and at a pre-selected distance apart from each other. The outer catheter 410 of the multi-window catheter system 400 may facilitate placement of the anchors at a pre-selected distance apart from each other. The outer catheter 410 may comprise any suitable number of openings 412, spaced apart by any suitable distance 418, as depicted in FIG. 5A. In one variation, the multi-window catheter may comprise 11-16 openings. Other variations may have about 5-10 openings while still other variations may have about 17-25 openings. Any suitable number of anchors may be delivered through each opening 412. In some variations, the method may comprise delivering one anchor through each opening. Thus, the number of openings in the outer catheter may correspond to the number of anchors secured in the ventricle. For example, in a variation where the multi-window catheter comprises 11-16 openings, 11-16 anchors may be delivered (one per opening). The distance between a first and last opening of the multi-window catheter may correspond to the approximate length of the implantable device when it is implanted (but before it is cinched). When one anchor is deployed through each opening in the multi-window catheter, the location and spacing of each anchor is determined by the spacing of the openings of the multi-window catheter. The openings 412 of the multi-window catheter 400 may be configured to facilitate the implantation a device that spans any suitable distance.

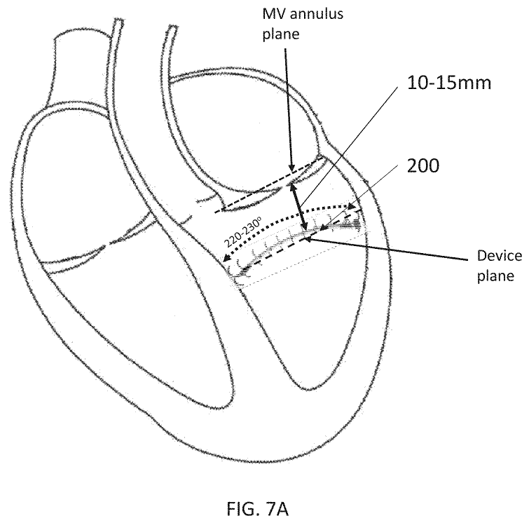

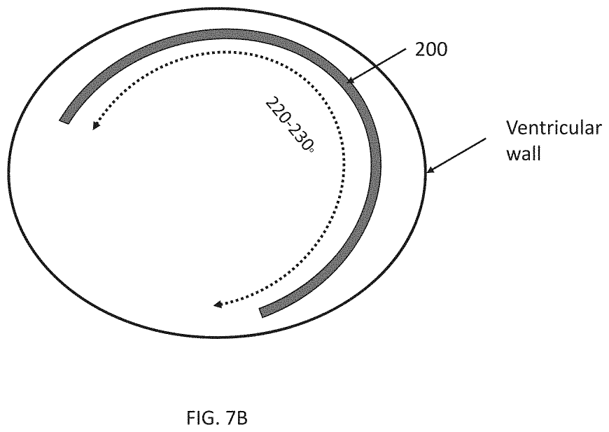

In a preferred variation depicted in FIGS. 7A and 7B, the method may comprise implanting the implantable device 200 to span approximately 220-230 degrees of a circumference or perimeter of the ventricle at a location of the implantable device. Methods may comprise implanting the device 200 to span the entire (or nearly the entire) free wall of the left ventricle, from the intersection of the septum to free wall under the mitral valve posterior leaflet P3 around to the left ventricle outflow tract under leaflet P1. Alternatively or additionally, implanting an implantable device that spans approximately 220-230 degrees of the circumference or perimeter of a region of the ventricle may comprise securing a cinchable device such that it spans at least about 55% or more (e.g., about 60% or more, approximately 60% to approximately 70%, approximately 61% to approximately 64%) of a circumference of the ventricle at the location of the implanted device. FIG. 7B depicts a view of the mitral valve from the left atrium, and as shown there, an implantable device may be configured to span at least about 55% or more of the circumference of the ventricle (e.g., at least about 220 degrees, approximately 220-240 degrees, and/or approximately two-thirds, e.g., about 66%, of the circumference of the ventricle (e.g., the widest portion of the ventricle), and/or spanning or subtending entirely or nearly entirely the free wall from the intersection of the septum to the MV posterior leaflet P3 to the LV outflow tract under leaflet P1) at the implantable device location. Methods of implanting an implantable device to span at least about 55% or more of the circumference of the ventricle at the implantable device location may comprise implanting an implantable device with a pre-cinch length of approximately 100-165 mm, depending upon the size of the left ventricle. It has been determined that a span of approximately 220-230 degrees (and/or at least about 55% of the ventricle circumference at the implantable device location) facilitates improved ventricle reshaping (e.g., reverse-remodeling) and reduction in mitral valve regurgitation. The number of anchors used in an implantable device that is implanted to span approximately 220-230 degrees may vary from patient to patient, and may vary based on the spacing of the anchors. For example, in some embodiments, the implantable device will comprise 11-16 anchors. The method of securing the implantable device to the ventricle wall may further comprise securing the implantable device such that a ratio R between the length of the implantable device and the diameter of the ventricle at the end of diastole is greater than about two. The length of the implantable device for purposes of calculating R is the length of the implantable device in an uncinched configuration, when the ventricle is at the end of diastole (i.e. at its widest point). Implanting the implantable device such that R is greater than about two may be accomplished by implanting the implantable device to span approximately 220-230 degrees of the ventricle. Alternatively or additionally, the implantation location of the cinchable device may be based on particular anatomical features or landmarks of the heart. For example, in one variation, the cinchable device may be secured to the left ventricle such that the device extends along a portion of the circumference of the ventricle between a junction of the septum and a ventricular free wall adjacent the mitral valve P3 leaflet, and a ventricular outflow tract.

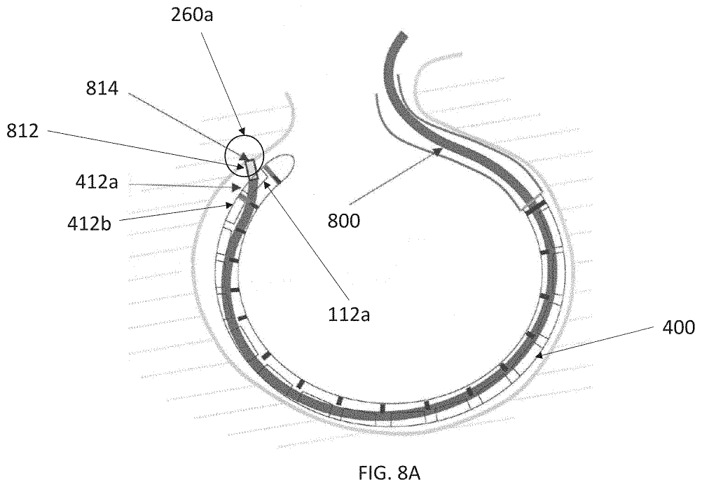

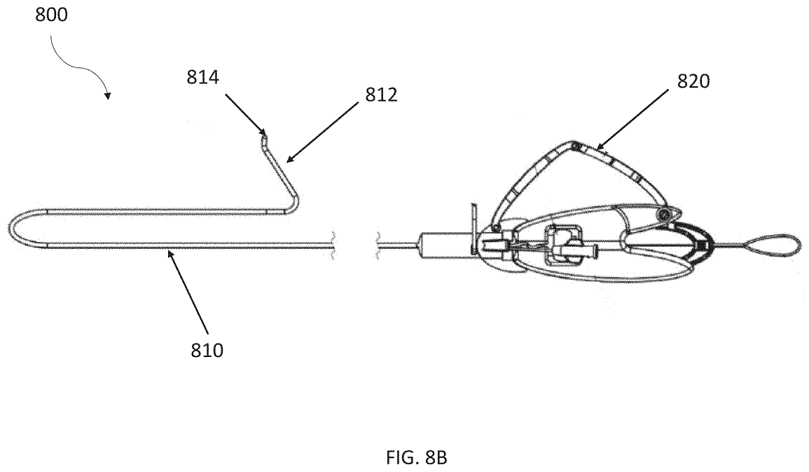

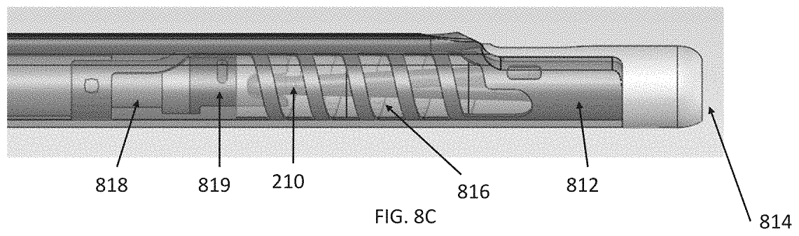

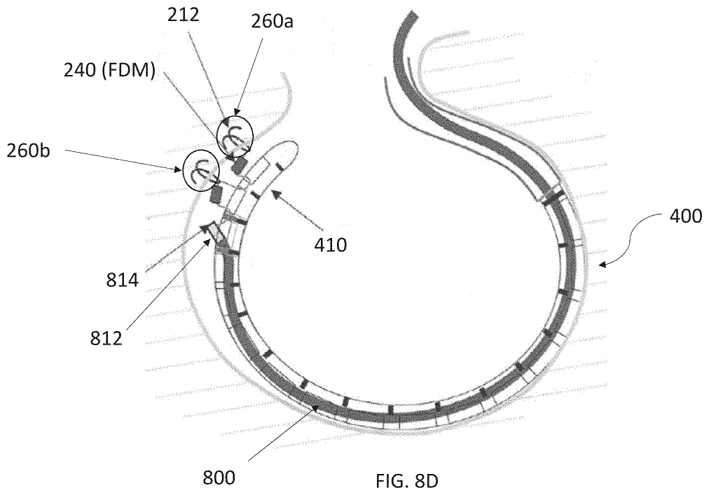

Securing the implantable device to the ventricular wall may further comprise extending an anchor delivery catheter 800 through the lumen of the multi-window catheter 400 to deploy one or more of the anchors of a plurality of tethered anchors, as depicted in FIG. 8A. FIG. 8B depicts one variation of an anchor delivery catheter 800 that may be used with any of the systems and methods described herein. The anchor delivery catheter 800 may comprise an elongate body 810 and one or more tissue anchors disposed within a first longitudinal lumen of the elongate body 810. The elongate body 810 may comprise a distal length 812 with a distal opening 814 in fluid communication with the first longitudinal lumen. A tissue anchor disposed within the first longitudinal lumen may exit the delivery catheter through the distal opening 814. FIG. 8C depicts a distal portion of the anchor delivery catheter 800, and shows an anchor 210 located in the longitudinal lumen 816 of the distal length 812 of the anchor delivery catheter. The anchor delivery catheter 800 may further comprise a push member 818 slidably disposed within the longitudinal lumen 816 and configured to contact and distally advance a tissue anchor 210, and a stop structure 819 located within the first longitudinal lumen and configured to restrict sliding the push member past a selected location along the first longitudinal lumen. The stop structure 819 may be a collet, band, ring, etc. As depicted in FIG. 8B, the anchor delivery catheter may further comprise a deployment handle 820 to actuate the push member to distally advance the tissue anchor 210 out of the lumen 816 of the delivery catheter 800. The anchor delivery catheter may further comprise a tissue depth indicator that may be slidable within a longitudinal lumen of the elongate body of the anchor delivery catheter. In some variations, a tissue depth indicator may comprise an elongate member such as a flexible wire that may be deflectable when apposed against tissue. The flexible wire may be secured to the outer surface of the anchor delivery catheter at a location that is proximal to the distal-most tip of the elongate body (i.e., the distal opening). The tissue depth indicator may comprise a first configuration that indicates the boundary of the surface of the target tissue location, and a second, bent or deflected configuration that indicates when the distal tip of the elongate body has been advanced to a preselected depth into the target tissue. The tissue depth indicator may provide the benefit of allowing the user to determine from fluoroscopic imaging whether the distal end of the anchor delivery catheter is sufficiently pressed into the tissue surface before deploying an anchor from within the catheter lumen into ventricular wall tissue. For example, it may be visible from a fluoroscopic image whether the depth indicator has transitioned between the first and second configurations, which may indicate whether the anchor delivery catheter is positioned appropriately to delivery an anchor to a target tissue location. Various features of anchor delivery catheters are described in U.S. Pat. Appln. Pub. No. 2016/0256149 which is hereby incorporated by reference in its entirety.

In one variation, securing the implantable device to ventricular wall tissue may comprise extending one or more anchor delivery catheters through one or more openings in a multi-window catheter and deploying the one or more anchors to one or more target implant sites, as depicted in FIGS. 8A and 8D. FIGS. 8A and 8D depict an anchor delivery catheter 800 extended through the lumen of a multi-window catheter 400, such that the distal length 812 of the anchor delivery catheter is extended through an opening of the multi-window catheter. In some variations, the method may comprise securing each of the plurality of anchors of the implantable device using a separate anchor delivery catheter. However, any suitable number of anchors may be delivered by a single anchor delivery catheter, e.g., a single anchor delivery catheter may deliver two or more anchors. Each anchor may have a target implant site in the ventricle wall that corresponds to a location of one of the openings of the multi-window catheter. In one variation, securing an implantable device to the ventricle may comprise actuating the inner catheter of the multi-window catheter to expose a first window of the outer catheter. The method may comprise extending a first delivery catheter 800 through the lumen of the multi-window catheter 400, as depicted in FIG. 8A. FIG. 8A depicts an anchor delivery catheter 800 extended through the lumen of a multi-window catheter 400 such that the distal length 812 of the anchor delivery catheter is extended through a first opening 412a of the multi-window catheter. The first window 412a may be the distal-most window of the multi-window catheter. Securing the implantable device may further comprise extending the distal length 812 of the anchor delivery catheter 800 through the unobstructed opening of the multi-window catheter 400 to a first target anchor location 260a of the ventricle wall. As described above, the one or more of the plurality of openings in the sidewall of the multi-window catheter may be obstructed by an inner catheter, such that only one opening is left unobstructed. Thus, the anchor delivery catheter may be guided to the first target implant site 260a of ventricle by the unobstructed opening 412a of the multi-window catheter. The method may further comprise advancing the distal length 812 of the first anchor delivery catheter 800 until it comes into contact with the first target implant site 260a on the ventricle wall, and deploying a first tethered anchor to the first target implant site 260a. Deploying the anchor may comprise actuating a deployment mechanism 820 of the anchor delivery catheter 800, as depicted in FIG. 8B. For example, deploying the tissue anchor may comprise advancing a push member to contact the anchor such that the anchor exists the distal opening of the delivery catheter. However, any suitable deployment mechanism may be utilized. Further methods and variations of deploying a tissue anchor are described in U.S. Pat. Appln. Pub. No. 2016/0256149 which is hereby incorporated by reference in its entirety.

FIGS. 8A and 8D depict an anchor delivery catheter 800 extending through a lumen of a multi-window catheter 400 to deliver anchor(s) to a target implant site. As depicted in FIG. 8D, a method of securing an implantable device into ventricular wall tissue may further comprise delivering a plurality of secondary anchors 214 to a plurality of target implant sites (e.g. 260a and 260b) in the ventricle wall. As described above, each anchor of an implantable device may be delivered sequentially by a different anchor delivery catheter. The method of securing a implantable device to a ventricular wall may further comprise loading a tether 220 attached to a first anchor 212 that has been secured to the ventricle wall into a secondary anchor delivery catheter (and/or a plurality of secondary anchor delivery catheters sequentially). For example, after a first tethered anchor 212 has been secured to the ventricle wall at a first target implant site 260a, the method may comprise securing a second anchor at a second target implant site 260b of the ventricle wall. Securing a second anchor may comprise loading the tether 220 into a second delivery catheter and threading the tether 220 through the anchor. The method may further comprise extending the second delivery catheter 800 through the lumen of the multi-window catheter 400. The method may further comprise advancing a distal portion of the second delivery catheter through a second opening 412b of the multi-window catheter 400 such that the distal portion of the second delivery catheter exits the second opening 412b and the distal opening 814 of the delivery catheter is positioned against the ventricular wall tissue at the target implant site. The method may further comprise actuating the deployment mechanism of the delivery catheter to deploy the anchor. This process may be repeated for each anchor of the implantable device, ending with the proximal-most, terminal anchor. The method may comprise extending the anchor delivery catheter through the distal-most opening of the multi-window catheter 400 to deliver the terminal anchor to a terminal target implant site of the ventricle. In some variations, between each anchor, the tether may be threaded into one or more FDMs 240 such that the anchors and FDMs are alternately deployed. For example, one or more FDMs may be loaded onto the tether before threading the tether into the next delivery catheter and through the next anchor. In some variations, the FDM(s) between the first, distal-most anchor and the second (next) anchor may include an uncoated FDM while the FDM(s) between the intermediate anchors (and optionally, the proximal-most, terminal anchor) may include a FDM that is coated with a polymer. In some variations, a FDM located between the first anchor (i.e., distal-most anchor) and the second anchor may be shorter than a FDM located between intermediate anchors, e.g., uncoated FDMs may be shorter than coated FDMs. Any of the methods described herein may alternatively comprise securing all of the anchors and FDMs of the implantable device simultaneously rather than sequentially as described above. For example, the method of securing an implantable device to a ventricle wall may comprise loading a plurality of anchors and FDMs of the implantable device into a single anchor delivery catheter. The anchor delivery catheter may be extended into the ventricle to simultaneously deliver the plurality of anchors to the ventricle wall. For example, the anchor delivery catheter may comprise a plurality of openings in a sidewall. Each opening may be configured to align with a target implant site of the ventricle wall when situated in the ventricle, similar to the opening of the multi-window catheter described above. The method may comprise loading the plurality of anchors into the lumen of the delivery catheter, and aligning each anchor with an opening. The method may further comprise advancing the delivery catheter into the ventricle, aligning the openings with the target implant site in the ventricle wall, and deploying the anchors simultaneously. The method may comprise actuating a push member to deploy all of the anchors simultaneously. However, any suitable mechanism may be used to deploy the anchors from the delivery catheter to the target implant locations.

### Cinching

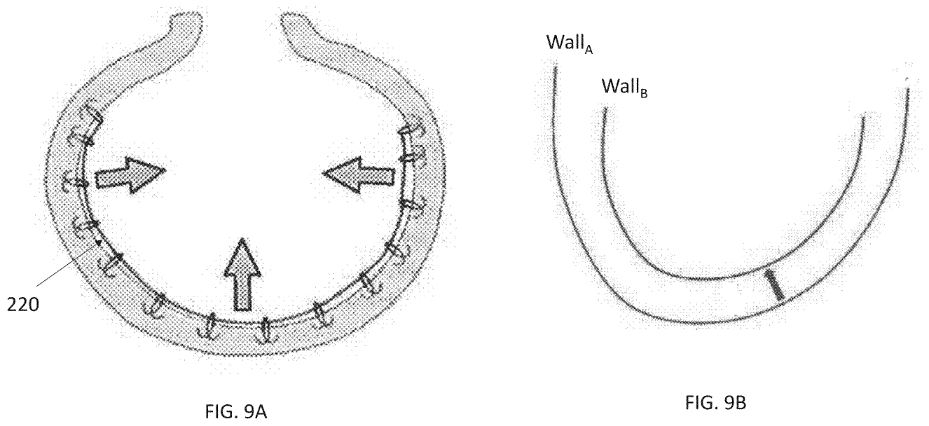

A method for reshaping (e.g., reverse-remodeling) the heart may further comprise cinching the tether of the implantable device to reduce the circumference of the ventricle wall at the location of the implantable device, as depicted in FIGS. 9A and 9B. FIG. 9A is a schematic representation showing the direction of the force exerted on the ventricle wall when the implantable device is implanted into ventricular wall tissue and is tensioned. FIG. 9B is a representation of how the ventricle wall is drawn inward (e.g., circumference reduced) at the location of the implantable device upon applying tension to the tether. In FIG. 9B, Walla represents the location of the ventricle wall boundary at a point in the cardiac cycle such as end-diastole, before the implanted device is cinched (i.e. when the device is in an uncinched configuration), and WallB represents the location of the ventricle wall boundary at the same point in the cardiac cycle, after the implanted device is cinched (i.e. when the device is in a cinched configuration). In some variations, cinching the implantable device to a cinched configuration may comprise applying tension to the tether 220. Tension may be applied to the tether 220 in any suitable fashion. Applying tension to the tether may have the effect of drawing the anchors of the implantable device closer together, thereby reducing the circumference of the ventricle wall at the location of the implantable device. The circumference of the ventricle wall may be reduced by any suitable amount. In a preferable variation, the method for reshaping a heart ventricle may comprise cinching the tether until the circumferential length of the portion of the ventricle where the implantable device is secured is reduced by approximately 30% (e.g., from about 25% to about 35%). Without wishing to be bound by theory, reducing the circumference of the ventricle wall at the location of the implantable device by about 30% may help provide a degree of dimension-reduction (e.g., reducing ventricular volume and/or circumference) while still providing sufficient freedom of movement for the ventricle to expand and contract. In order to consistently secure the implantable device in a cinched configuration such that the circumference of the ventricle at the location of the implantable device is reduced by approximately 30%, the method may comprise cinching the implantable device to a hard stop configuration, and subsequently securing a lock member to the implantable device at a pre-determined distance from the terminal anchor of the implantable device, as described below. While the examples described and depicted herein use a cinchable device comprising tethered anchors, various cinchable devices may be used to reduce the ventricle by 30% at a location approximately 10-15 mm below the mitral valve (e.g. devices comprising clips, shape memory material, or springs secured to tissue and coupled to a sheath which may be contracted to place tension on the springs).

### Locking

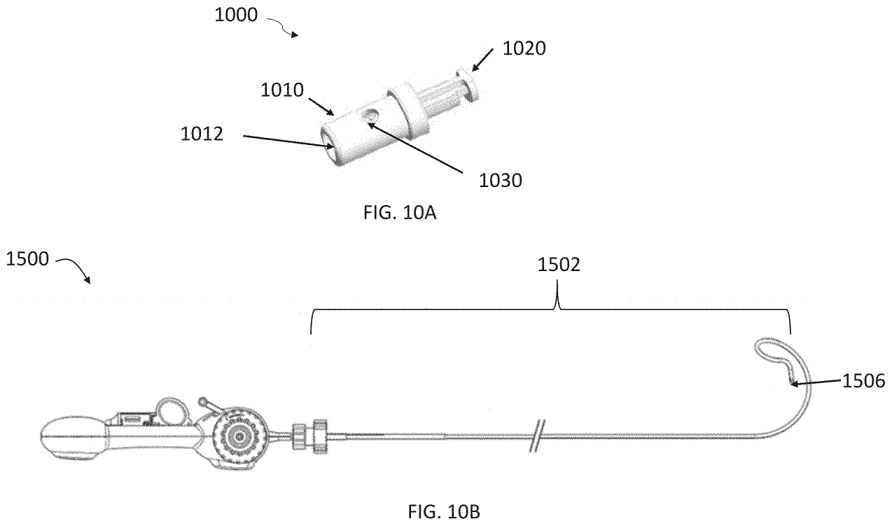

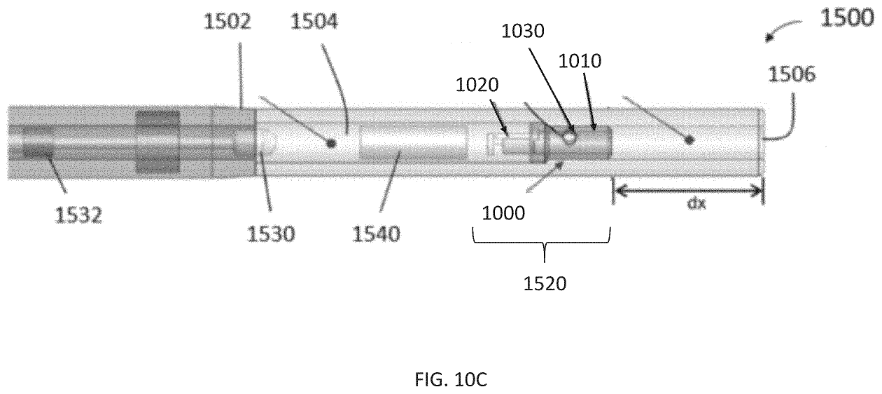

Methods of reshaping (e.g., reverse-remodeling) a heart ventricle may comprise locking the implantable device in a cinched configuration, i.e., to retain the tension in the tether such that the implantable device provides a persistent cinching effect and/or compression on the ventricular wall tissue, leading to a dimensional and volumetric reduction of the ventricle. FIG. 10A depicts one variation of a lock member and FIGS. 10B-10C depict one variation of a combined tether cinching/tensioning and lock member deployment catheter that may be used to lock the implantable device in a cinched configuration. Locking the implantable device in a cinched configuration may comprise securing a lock member onto the tether proximal to the terminal anchor of the implantable device. Securing the lock member onto the tether may comprise threading the tether into a lumen of the lock deployment catheter 1500 and through an opening in the lock member 1000, and advancing the lock member delivery catheter to the location of an implantable device in the ventricle. Once the lock member deployment catheter is advanced to the desired location, the method may comprise applying tension to the tether to cinch the implanted device. Once the device is in a cinched configuration, the method may comprise actuating the lock to secure it to the tether, then releasing the lock from the lock member delivery catheter. For example, the lock member deployment catheter may be advanced over the tether to the terminal anchor of the implantable device, the lock member may be secured onto the tether, and then released from the docking section of the deployment catheter. In some variations, the method may comprise securing the lock member at a pre-determined distance from the terminal anchor. The lock member may be secured such that it is fixedly attached to the tether (i.e., not slidable on the tether). Fixedly securing the lock member at a predetermined distance from the terminal anchor may provide the benefit of introducing a pre-determined amount of slack into the tether. Securing the lock member at a pre-determined distance from the terminal anchor may allow for some flexibility in the implantable device, which may help improve cardiac function and ventricle reshaping by allowing a greater range of motion of the ventricular wall as the ventricle expands and contracts during diastole and systole.

The lock member used to retain the device in a cinched configuration (i.e., by retaining the tension on the tether) may be any suitable suture lock member. FIG. 10A depicts an exemplary variation of a lock member 1000 comprising a tube 1010 and a plug 1020 configured to fit within a lumen 1012 of the tube 1010. The tube and/or plug may comprise one or more openings 1030 for the passage of the implant tether though the lumen. The opening 1030 may be located along a side wall of the tube 1010. To deploy the lock 1000, the plug 1020 may be pushed into the tube 1010 to clamp the tether 1020 between the walls of the lock plug 1020 and tube 1010, thereby securing the lock member to the tether. In some variations, the tension on the tether may cause the plug 1020 to rotate (e.g., in a direction that is perpendicular to the longitudinal axis of the plug) and further increase the engagement of the lock member on the tether. The method of reshaping a heart ventricle may comprise securing the lock member to the tether proximal to the terminal anchor to secure the implantable device in a cinched configuration. The lock may be secured on the tether by friction fit, snap fit, screw fit, and/or any other suitable mechanism. The lock member 1000 may be made of any suitable material. For example, the lock member may be comprised of nitinol, plastic, polymer, metal, or any other suitable material. Further, the lock member may be comprised of more than one material. For example, the plug 1020 portion may be comprised of a different material than the tube 1010 portion of the lock member 1000. Although methods described herein describe using a lock member to secure the implantable device in the cinched configuration, any suitable mechanism may be used to secure the implantable device in a cinched configuration. For example, in another variation, the tether may be secured directly to the terminal anchor. Variations on lock members are further described in U.S. Pat. Appln. Pub. No. 2010/0121349 which is hereby incorporated by reference in its entirety.

FIGS. 10B-10C depict one variation of a lock deployment catheter 1500 that may be used to secure a lock member onto a tether to lock the device in a cinched configuration. The lock deployment catheter 1500 may comprise an elongate body 1502 with a longitudinal lumen 1504 that terminates at a distal opening 1506, a lock member 1000 located at a lock member docking section 1520 in the lumen, and a push member 1530 within the longitudinal lumen. Advancing the push member 1530 distally may contact and urge the lock member 1000 distally within the lumen 1504 and through the distal opening 1506. The lock deployment catheter 1500 may also comprise a push member stop member 1540 within the longitudinal lumen to limit distal advancement of the push member 1530. The stop member 1540 may have a lumen through which a portion of the push member may pass. The push member 1530 may comprise stop tube 1532 located along the length of the push member that has a greater diameter than the stop member 1540 such that distal advancement of the push member is blocked when the stop tube 1532 contacts the stop member 1540. The stop member 1540 may be a collet, band, ring, etc., that is secured to the internal surface of the lumen 1504. Alternatively, the stop member 1540 may be a region of the lumen with a diameter that is less than the diameter of the stop tube (e.g., a narrowed portion of the lumen).

In some variations, the lock member deployment catheter may be configured to introduce a pre-determined amount of slack to the tether by deploying the lock member at a pre-determined distance from the terminal anchor. The lock deployment catheter 1500 is configured to secure the lock member at a pre-selected distance (i.e., a lock distance offset doffset) away from the terminal anchor so that a corresponding pre-selected amount of slack is provided to the implant. For example, the lock docking section 1520 may be offset from the lock exit opening by a pre-selected offset (dx) so that when the lock is secured on the tether, the lock is located at the pre-selected offset from the lock exit. The lock distance offset doffset may be the sum of the pre-selected offset (dx) and the distance from the lock exit to the terminal anchor. In the case where the lock exit is at the distal-most end of the deployment catheter and the distal-most end is in direct contact with the terminal anchor, the lock distance offset doffset may be approximately the same as the pre-selected offset (dx). The distance or offset (dx) away from the distal-most opening 1506 may be, for example, from about 5 mm to about 15 mm, e.g., from about 6 mm to about 11 mm, from about 8 mm to about 10 mm, about 7 mm, about 9.5 mm, etc. The lock member may be retained in the docking section by any releasable mechanism, for example, by friction-fit, snap-fit and/or a frangible connection.

Introducing a pre-determined amount of slack may comprise cinching the implantable device to a hard stop configuration prior to securing the lock member onto the tether. Cinching the device to a hard stop configuration may comprise applying tension to the tether until the implantable device cannot be cinched any further because the components of the implantable device (i.e. anchors and FDMs) are in contact with each other. When cinched to its hard stop, the implant may become incompressible. That is, the implant can bend, but its perimeter cannot be further reduced. Cinching the implantable device to a hard stop configuration before introducing a pre-determined amount of slack into the implantable device may help introduce a predictable and repeatable amount of slack into the implantable device. Providing a pre-selected length of tether to an implant that has been cinched to its hard stop configuration may comprise securing a lock member situated at a pre-determined distance from the distal end of the lock deployment catheter to the tether, thereby securing the lock member at a pre-determined distance from the terminal anchor. Introducing a pre-determined amount of slack to the tether may provide the benefit of allowing for consistent cinching of the implantable device by a prescribed amount. For example, as described above, it may be desirable to cinch the implantable device such that the portion of the ventricle circumference at the location of the implantable device is reduced by 30% (e.g., from about 25% to about 35%). Lock deployment catheters may be configured to secure a lock member to a tether at a distance from the terminal anchor that provides a 30% reduction in ventricle circumference at the location of the implantable device. For example, a distance (dx) between the lock docking section (i.e., the location along the lock deployment catheter where the lock member is secured onto the tether) and the lock exit opening (e.g., opening at the distal end of the lock deployment catheter) may be increased or decreased according to the desired amount of slack and/or ventricle circumference reduction. In some variations, the distance between the lock docking section and the lock exit opening may be from about 5 mm to about 15 mm, e.g., about 9.5 mm, to attain a pre-determined amount of slack that provides about a 30% reduction in ventricle circumference at the location of the implantable device. If a greater circumference reduction is desired (e.g., from about 35% reduction to about 40% reduction), the distance between the lock docking section and the lock exit opening may be from about 0 mm to about 5 mm, e.g., about 3.5 mm. Alternatively or additionally, a greater circumference reduction may be attained by using shorter FDMs. If a smaller circumference reduction is desired (e.g., from about 10% reduction to about 20% reduction), the distance between the lock docking section and the lock exit opening may be from about 20 mm to about 35 mm, e.g., about 25 mm. Alternatively or additionally, a smaller circumference reduction may be attained by using longer FDMs.

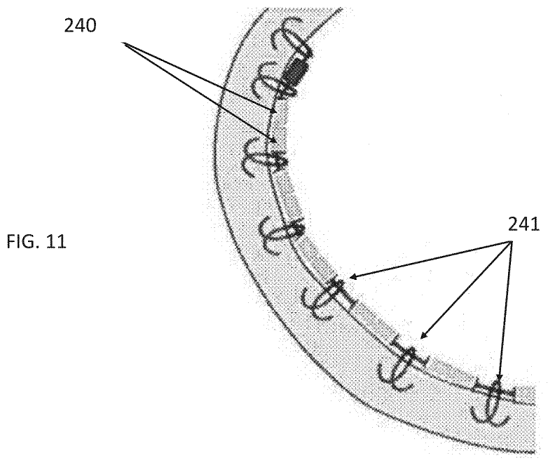

Introducing a pre-determined amount of slack into the implantable device may provide the benefit of allowing for a greater degree of left ventricle wall motion during systole, which may help facilitate ventricular reshaping (e.g., reverse-remodeling). That is, the implantable device may be more “flexible” and allow for a greater degree of ventricular wall motion. FIG. 11 depicts an implantable device comprising a plurality of tethered anchors and FDMs secured to a ventricle wall with a pre-determined amount of slack. Introducing the pre-determined amount of slack allows for gaps 241 between the FDMs and/or anchors. When very little slack is provided to the implant, the distance between intermediate FDMs 240 may be nearly zero. For example, if the lock is secured immediately adjacent to the proximal-most terminal anchor (i.e., the lock member is secured onto the tether about 0 mm away from the proximal-most terminal anchor), little if any slack is provided to the implant and ventricular wall motion may be limited or constrained in a manner that does not promote therapeutic cardiac remodeling. Additional details regarding various FDMs that may be used between any of the anchors in any of the implantable devices described herein are provided in U.S. patent application Ser. No. 15/817,015, filed Nov. 17, 2017, which is hereby incorporated by reference in its entirety.

### Systems and Kits

Also described herein are systems for implanting devices for reshaping (e.g., reverse-remodeling) a heart ventricle. Systems for implanting a device may comprise a multi-window catheter, one or more anchor delivery catheters, an implantable device comprising a plurality of anchors coupled to a tether, a lock member, and a lock member deployment catheter. The implantable device may be any of the implantable devices described above. In some variations, the implantable device may comprise one or more FDMs that are slidably coupled to the tether and disposed between the anchors. For example, as described in reference to FIGS. 2 and 3, an implantable device may comprise a plurality of tethered anchors, and force distribution members coupled to the tether and situated between the anchors. Systems described herein may comprise one or more anchor delivery catheters that may be configured to secure each anchor of the device into the tissue of the ventricle wall, thereby securing the device to the ventricle. For example, a system comprising an implantable device having 11-16 anchors may comprise 11-16 anchor delivery catheters where each anchor delivery catheter houses and/or delivers a single anchor. Alternatively or additionally, the system may comprise anchor delivery catheters that may be configured to house and/or delivery multiple anchors. Systems described herein may comprise a multi-window catheter configured to position a device at a described location, for example, at a location in the ventricle approximately 10-15 mm below the mitral valve annulus as depicted in FIG. 6. In some variations, a multi-window catheter may comprise a pre-defined curvature that approximates the curvature at the widest point of the ventricle and/or the region of the ventricle that is about 10-15 mm below the mitral valve annulus. Some variations may comprise a multi-window catheter may comprise a reinforced distal curvature to facilitate implantable device placement in a particular plane (e.g., a plane that is approximately parallel to a plane of the mitral valve annulus), as depicted in FIG. 6. In some variations, a system may comprise an implantable device configured to span approximately 220-230 degrees of the circumference of the ventricle (e.g., approximately two-thirds of the ventricle circumference at the location of the implantable device, at least about 200 degrees or more, subtending the free wall from the intersection of the septum to the MV posterior leaflet P3 to the LV outflow tract under leaflet P1). For example, a multi-window catheter may comprise side wall openings that are spaced with a distance such that the implantable device may span approximately 220-230 degrees of the ventricle, as depicted in FIG. 7.

In some variations, a system may comprise a lock member and lock deployment catheter configured to lock the implantable device in a desired cinched configuration. For example, as described above and depicted in FIG. 10C, a system may comprise a lock member deployment catheter configured to secure a lock member at a predetermined location from a terminal anchor of the implantable device.

### Experimental Results

Patient studies were conducted to evaluate the efficacy of devices and methods described herein as compared to alternative devices and methods. Each experiment/study was conducted using a cinchable device comprising a plurality of anchors coupled to a tether, with FDMs between successive anchors, as described above. The cinchable devices were implanted into the left ventricle of patients. Various cardiac function metrics were measured prior to implantation and at various time points after implantation (e.g., 30 days, 60 days, 3 months, 6 months, a year, etc.) to monitor changes and improvements in cardiac function. Cardiac function metrics that were measured included ejection fraction percentage, left ventricle end systolic volume, left ventricle end diastolic volume, New York Heart Association Classification, and others.

Cardiac function data/metrics were collected for three groups of patients, where each patient group had a cinchable device implanted in their left ventricles using different implantation methods. In all groups, the cinchable device comprised a plurality of tissue anchors and FDMs coupled to a tether, and the cinchable device was implanted in the left ventricle.

The first group of patients had the cinchable device implanted according to the methods described herein (e.g., as represented by the flowchart in FIG. 1, hereinafter “Method 1”). The steps of Method 1 are as follows. The cinchable device was implanted approximately 10-15 mm below the mitral valve in a plane approximately a parallel to the plane of the mitral valve and secured to the ventricle such that the cinchable device spanned approximately 220-230 degrees or about two-thirds of the circumference of the ventricle wall (e.g., the widest portion of the ventricle) at the implantable device location (e.g., at least about 200 degrees or more, subtending the free wall from the intersection of the septum to the MV posterior leaflet P3 to the LV outflow tract under leaflet P1). A multi-window catheter comprising a reinforced curvature at a distal end was used to implant the device into the ventricle over substantially the entire free wall of the LV such that the ratio “R” between the uncinched length of the implantable device and the inner diameter of the ventricle at end-diastole has a magnitude greater than or equal to about 2. Fluoroscopic imaging was used to confirm the location of the device and ensure that the device was placed in a plane substantially parallel to the plane of the mitral valve. The implantable device was then cinched such that the circumference of the ventricle wall at the implantable device location decreased by approximately 30% (e.g., from about 25% to about 35%) with the introduction of a pre-determined amount of slack. The average circumferential reduction (i.e., the average cinch) was about 32% and the average value of the ratio R was about 2.05 for this first group of patients. The lock was then actuated, and a predetermined amount of slack was introduced onto the tether automatically as the lock was released from its catheter, thereby locking the device in a cinched configuration. The predetermined amount of slack was introduced into the device using a lock deployment catheter with a lock member offset from a distal opening of the lock deployment catheter by a predetermined amount. Introducing a measure of slack to the device may result in a relative more flexible device (e.g., as compared to a device that cinched to a hard stop without any slack), which may help the device accommodate the motion of the walls of the beating heart.

The second group of patients had the cinchable device targeted to 10 mm below the mitral valve annulus and the length of the implant was shorter than Method 1 implants, such that and the ratio R was less than 2 (hereinafter “Method 2”). The cinchable device was implanted in a variety of orientations relative to the plane of the mitral valve (e.g., cinchable implants were implanted at varying angles relative to the mitral valve plane). The length of the FDMs of the devices implanted in the second group were longer than the FDMs of the devices used in Method 1 above. The cinchable device was cinched to a hard stop configuration, such that the circumference of the ventricle wall at the location of the implantable device was reduced by about 20%. No slack was fed back into the implant, such that the implant was more rigid, and less flexible than the Method 1 implants. The average circumference reduction (i.e., the average cinch) was about 19% and the average value of the ratio R was about 1.8 for this second group of patients. The device was then locked in the cinched configuration, with little (if any) any slack, i.e., locked in the hard stop configuration. Without any additional slack, the device may be more rigid (i.e., less flexible) than a device where a pre-determined amount of slack was introduced prior to locking the device.

The length and orientation of the implants of the third group of patients were the same as Method 2 patients, but the cinchable device was cinched to a hard stop configuration and then a predetermined amount of slack was introduced before locking the device (hereinafter “Method 3”). The device was locked in a cinched configuration using a lock-deployment catheter as described above. The predetermined amount of slack was introduced into the device using a lock deployment catheter with a lock member offset from a distal opening of the lock deployment catheter by a predetermined amount. The average circumference reduction (i.e., the average cinch) was about 21% and the average value of the ratio R was about 1.39 for this third group of patients.

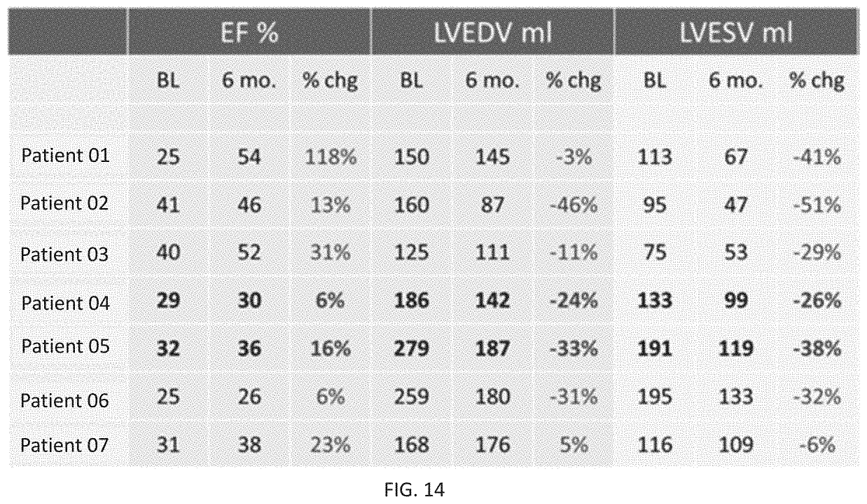

As supported by the data in FIGS. 14-23, implantable devices implanted according to Method 1 resulted in improved cardiac function compared to other implantable devices and methods (e.g., Method 2 and Method 3).

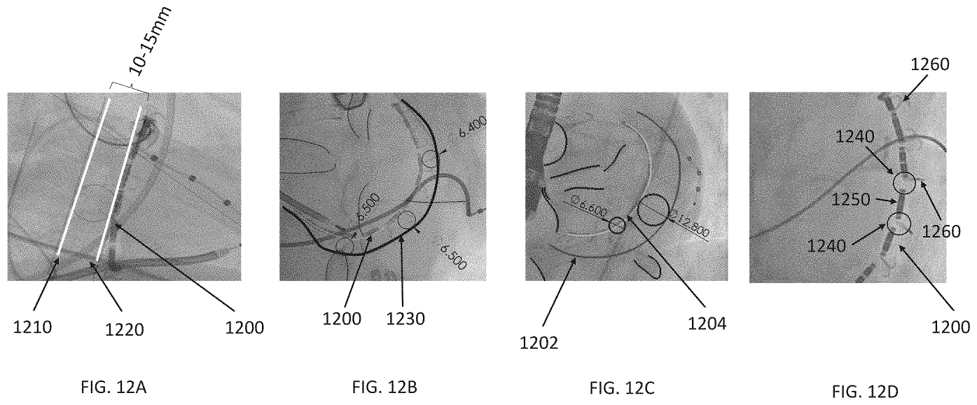

FIGS. 12A-12C depict a series of fluoroscopic images of cinchable devices that were implanted into the left ventricles of patients according to Method 1. FIG. 12A is a side view of the ventricle, depicting the cinchable device 1200, the plane of the mitral valve annulus 1210, and the plane of the cinchable device 1220. As depicted in FIG. 12A, the cinchable device 1200 was secured to the left ventricle at a location approximately 10-15 mm below the mitral valve, and in a plane 1220 substantially parallel to the plane of the mitral valve 1210. FIGS. 12B and 12C are images of the ventricle as viewed from below (i.e., from the apex of the LV), depicting the cinchable device 1200 in cinched configurations. FIG. 12B depicts the cinchable device 1200 implanted in the ventricle wall, in the cinched and uncinched configurations. The contour line 1230 represents the location and span of the cinchable device after it was implanted but before it was cinched, i.e., in its uncinched configuration. The cinchable device 1200 was implanted such that it spanned approximately 220-240 degrees of the ventricle wall at the location of the cinchable device (e.g., approximately two-thirds of the ventricle circumference at the location of the implantable device, approximately 61% to approximately 64% of the ventricle circumference at the location of the implantable device, at least about 200 degrees or more, subtending the free wall from the intersection of the septum to the MV posterior leaflet P3 to the LV outflow tract under leaflet P1), and such that the ratio R between the uncinched length of the cinchable device and the inner diameter of the ventricle at end-diastole had a magnitude greater than about 2. After the anchors of the cinchable device were secured to the ventricle wall, the cinchable device was cinched, and secured in a cinched configuration. FIG. 12C depicts the cinchable device in an uncinched configuration 1202, and in a cinched configuration 1204. As depicted in FIG. 12C, the cinchable device was cinched such that the circumferential portion of the ventricle at the location of the cinchable device was reduced by approximately 30% (e.g., from about 25% to about 35%). After cinching, the cinchable device was locked in the cinched configuration.

FIG. 12D is an image of a portion of a cinchable device 1200 that was implanted in the ventricle in accordance with Method 3, with a pre-determined amount of slack introduced. As seen in the fluoroscopic image, the additional slack allows for gaps 1240 to be formed between the FDMs 1250 and anchors 1260. These gaps 1240 indicate that there is space in the implantable device to allow for contraction of the ventricle during systole. Introducing a pre-determined amount of slack into the implantable device to provide a desired amount of flexibility was accomplished by securing a lock member from about 8 mm to about 12 mm away from the terminal anchor of the implantable device, e.g., about 9 mm, about 9.5 mm, about 10 mm, about 10.5 mm, about 11.25 mm, about 11.75 mm, about 12 mm.

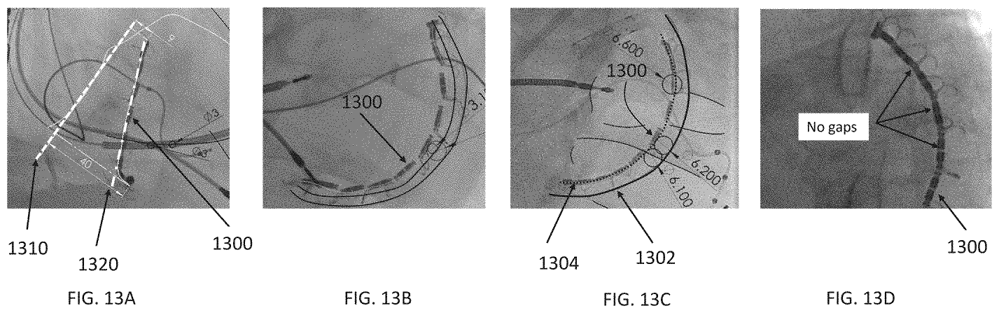

FIGS. 13A-D are a series of fluoroscopic images of cinchable devices implanted according to Method 2. FIG. 13A depicts a side view of a ventricle with a cinchable device 1300 implanted into the ventricle according to Method 2. The cinchable device 1300 was implanted near the mitral valve and was not implanted with any particular orientation to the mitral valve, and in this patient, the cinchable device plane 1320 is not parallel to the mitral valve plane 1310. FIG. 13B is a bottom view of the ventricle (e.g., from the apex of the LV) depicting a cinchable device 1300 implanted in the ventricle wall in its cinched configuration. The cinchable device in FIG. 13B was implanted in the ventricle wall such that the ratio R of the uncinched length to the diameter of the ventricle is less than two. FIG. 13C depicts the cinchable device in its cinched configuration. The contour line 1302 represents the location and span of the cinchable device 1300 after it was implanted but before it was cinched, i.e., in its uncinched configuration, while the contour line 1304 represents the location and span of the cinchable device in its cinched configuration. As may be seen in the fluoroscopic image of FIG. 13C, the cinchable device was cinched such that the circumferential portion of the ventricle at the location of the cinchable device was reduced by approximately 20%. After cinching, the cinchable device 1300 was locked in a cinched configuration. In Method 2, the cinched configuration is the hard stop configuration, where no slack was provided to the tether when the cinchable device was locked. FIG. 13D depicts the implantable device 1300 secured to the ventricle wall such that no gaps were visible between the FDMs and the anchors.

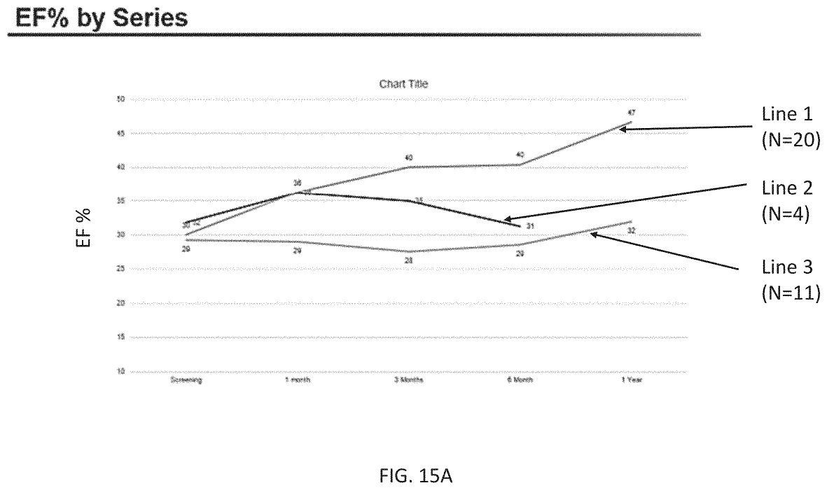

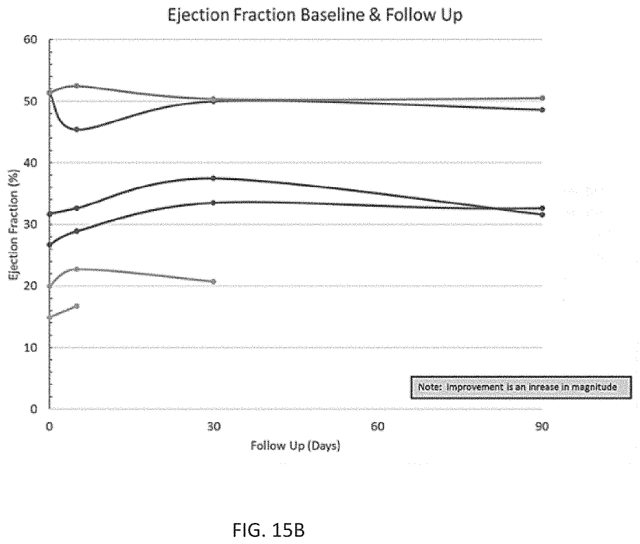

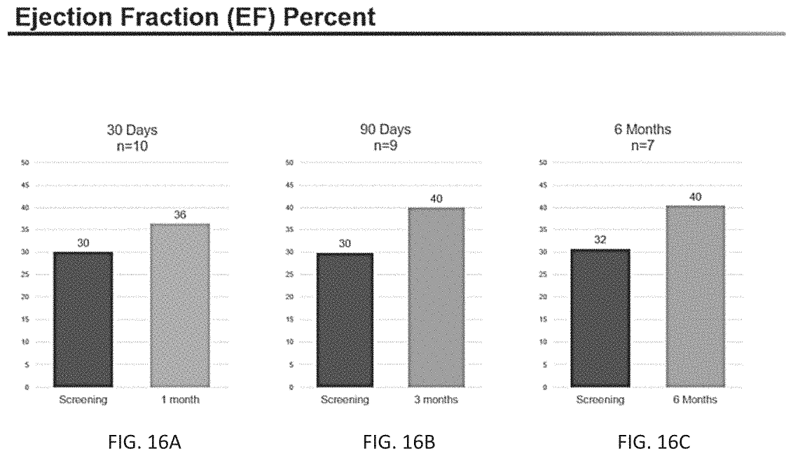

Data indicative of cardiac function was collected for patients treated according to Methods 1, 2 and 3 at various time points. In studies evaluating the efficacy of Methods 1, 2, and 3, patients were monitored for a year post-implantation of the implantable, cinchable device, and data was collected for multiple cardiac function metrics. FIG. 14 is a table summarizing the results for patients treated according to Method 1. The chart in FIG. 14 depicts ejection fraction percentage (EF %), left ventricle end diastolic volume (LVEDV), and left ventricle end systolic volume (LVESV) at baseline (i.e., prior to implantation according to Method 1) and 6 months, and indicates the percentage change in each metric for seven patients. The differences in each metric between baseline and six months show a trend towards improvement in EF %, LVEDV, and LVESV, as discussed in further detail below. Data was also collected for patients treated according to Methods 2 and 3. For Methods 2 and 3, patients were monitored for one year post-implantation of an implantable cinchable device, and data was obtained for multiple metrics of cardiac function at various time points. FIGS. 15-23 depicts various representations of measurements taken over time of multiple metrics of cardiac function for patients treated with an implantable, cinchable device according to Methods 1, 2, and 3. Specifically, FIG. 15A shows EF % over time for patients treated according to Method 1 (Line 1), Method 2 (Line 2), and Method 3 (Line 3). FIGS. 16A-16C, 18A-18C, 19A-19C, 20A-20C, and 22A-22C show various cardiac function metrics measured over time for patients treated according to Method 1. FIGS. 15B, 17A-17C, 21A-21C, and 23A-23C show various cardiac function metrics measured over time for patients treated according to Method 2. Taken together, FIGS. 15-23 demonstrate that Method 1 exhibited improved cardiac function compared to Method 2 as measured by performance metrics such as EF %, LVESV, LVEDV, and NYHA Class.

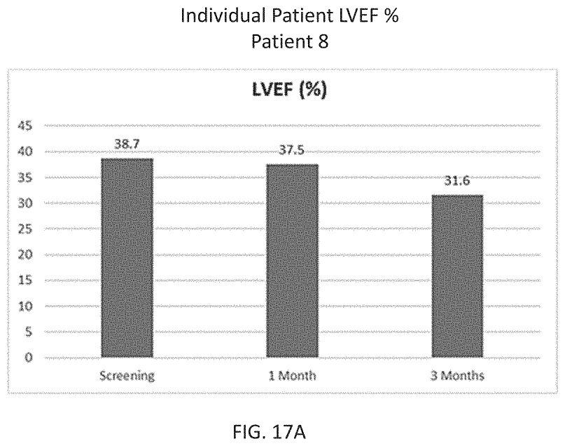

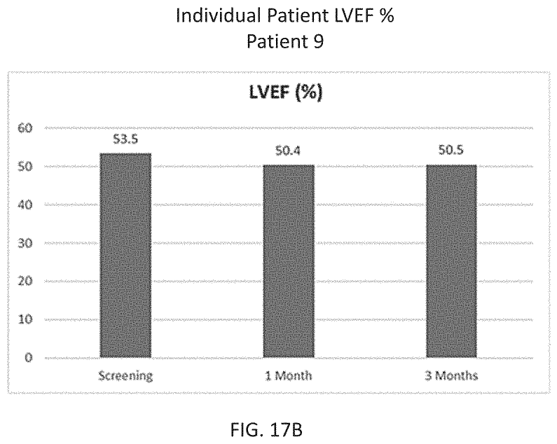

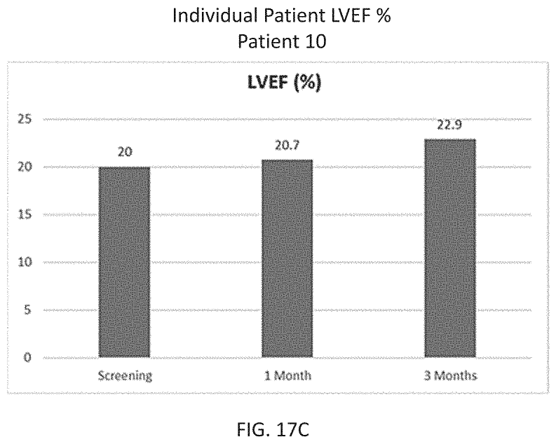

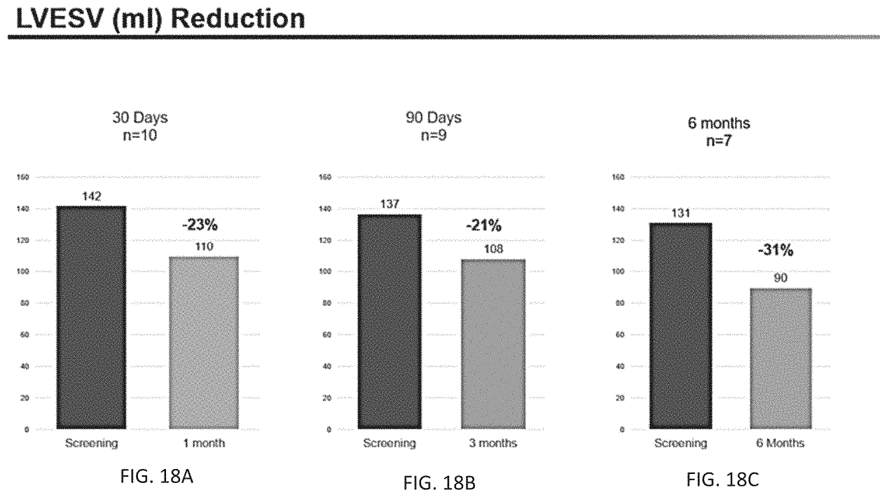

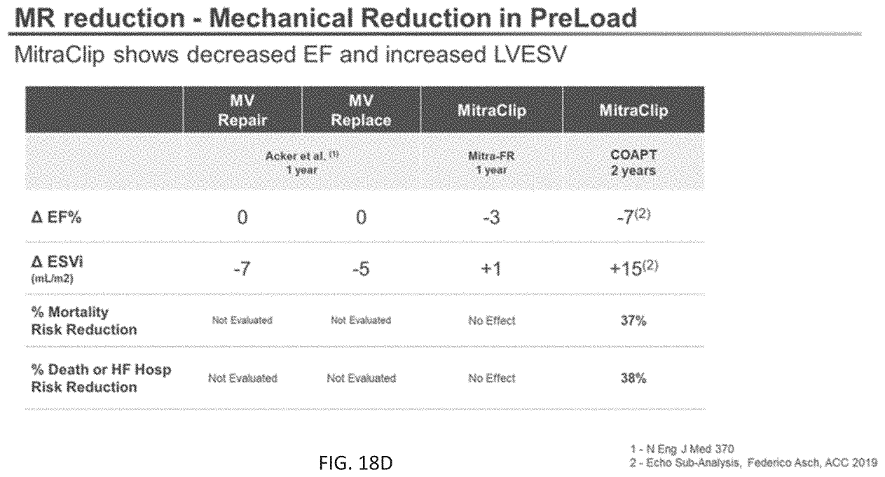

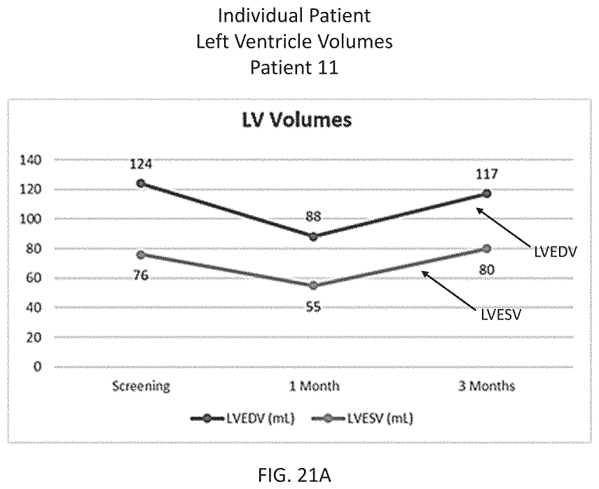

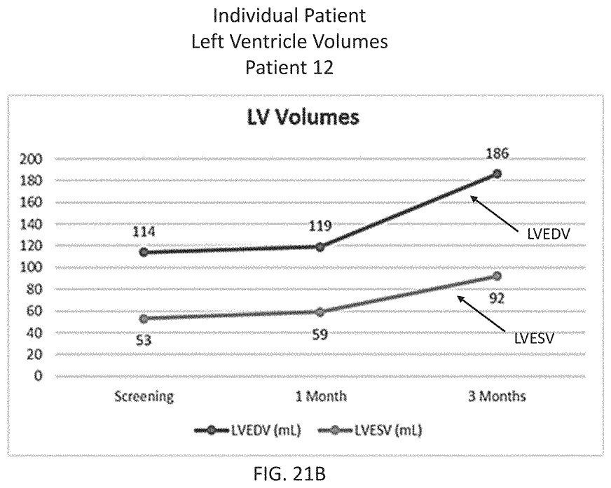

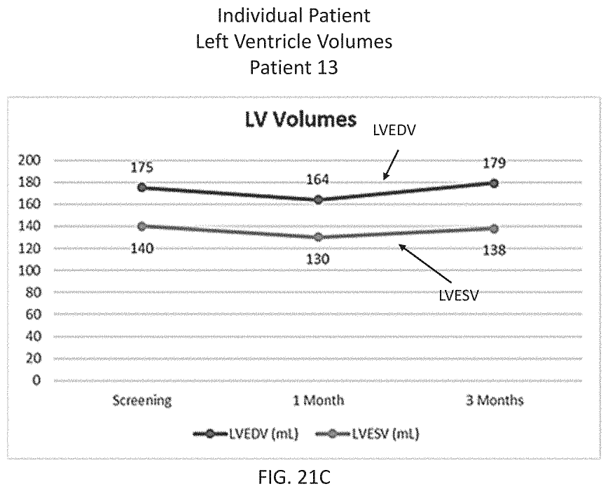

FIG. 15A depicts ejection fraction percentage over time for Method 1 (Line 1), Method 2 (Line 2), and Method 3 (Line 3). This plot shows that the systems and methods disclosed herein (i.e., Method 1) were more effective at facilitating the therapeutic remodeling of the ventricle, as well as the reduction of mitral valve regurgitation, as compared other cinchable devices and methods (i.e. Methods 2 and 3). FIGS. 16A-C demonstrate improved ejection fraction over time for patients treated according to Method 1, whereas FIGS. 17A-C demonstrate little to no improvement in ejection fraction over time for individual patients treated according to Method 2. FIGS. 18A-C, 19A-C, and 20A-C demonstrate reductions in LVESV, LVESD, and LVEDV, respectively, over time for patients treated according to Method 1. In contrast to the improvements in cardiac function when treated using Method 1, FIGS. 21A-C show little to no improvement in LVESV and LVEDV over time in patients treated according to Method 2. These metrics indicate improved cardiac function using Method 1 compared to Method 2, and therefore suggest that Method 1 was more effective in facilitating therapeutic remodeling of the ventricle.

FIG. 15A is a plot of Ejection Fraction percentage (EF %) over time for patients treated according to experimental Methods 1, 2, and 3. Ejection fraction percentage is the percentage of blood that is pumped out of a filled left ventricle with each heartbeat, and is one of multiple metrics for evaluating the function of a heart. Generally, an ejection fraction percentage of 55-70 percent is normal for a healthy heart. Line 1 represents EF % measured at the time of screening and at 1 month, 3 months, 6 months, and 1 year after implantation of a cinchable device according to Method 1. Line 2 represents EF % measured at the time of screening and at 1 month, 3 months, 6 months, and 1 year after implantation of a cinchable device according to Method 2. Line 3 represents EF % measured at the time of screening and at 1 month, 3 months, 6 months, and 1 year after implantation of a cinchable device according to Method 3. Lines 1, 2, and 3 demonstrate that EF % increased more for patients treated according to Method 1 (Line 1), as compared to the EF % for patients treated according to Methods 2 and 3 (Lines 2 and 3, respectively).

As shown in FIG. 15A, patients with a device implanted according to Method 1 exhibited a higher ejection fraction percentage than the patients with device implanted according to Methods 2 and 3. The increased EF % values for patients with devices implanted according to Method 1 compared to patients with devices implanted according to Methods 2 and 3 demonstrates improvement in cardiac function. These results suggest that implantation of cinchable devices according to Methods 2 and 3 stimulate little if any therapeutically or functionally significant ventricular modelling. For example, FIG. 15B depicts EF % data for six individual patients that have been treated according to Method 2. Each line in FIG. 15B represents the EF % value of an individual patient prior to implantation (0 days) up to 90 days after implantation. This data demonstrates that EF % did not significantly improve over time for the individual patients. Surprisingly, Method 1, where the cinchable device was implanted well-below the mitral valve and locked with a predetermined amount of slack (e.g., not cinched and locked to a hard stop), resulted in significantly greater cardiac function improvement than methods that implanted the cinchable device in close proximity to the mitral valve and cinched to a hard stop (e.g., Method 2).

FIGS. 16A-16C depict a series of bar graphs that further demonstrate the improved cardiac function measured using the methods described herein (i.e., Method 1). FIGS. 16A-C show average EF % for multiple patients at one, three, and six months after device implantation (according to Method 1) as compared to the ejection fraction percentage at screening (i.e., prior to device implantation). FIG. 16A depicts average EF % for 10 patients at screening versus average EF % at one month post-implantation. The average EF % increased from screening to one month by 6% for these 10 patients. FIG. 16B depicts average EF % for 9 patients at screening versus average EF % at three months post-implantation. Average EF % increased from screening to three months by 10% for these 9 patients. FIG. 16C depicts average EF % for 7 patients at screening versus average EF % at six months post-implantation. Average EF % increased from screening to six months by 8% for these 7 patients.

FIGS. 17A-17C depict a series of bar graphs showing ejection fraction percentage at screening, 1 month post-implantation, and 3 months post-implantation for individual patients that were treated according to Method 2. FIG. 17A shows a decrease in EF % from 38.7% to 31.6% between screening and three months post-implantation for Patient 8. This decreased EF % indicates that the cardiac function of Patient 8 decreased after implantation of the device according to Method 2. FIG. 17B shows a decrease in EF % from 53.5% to 50.5% between screening and three months post-implantation for Patient 9. This decrease in EF % indicates that the cardiac function of Patient 9 decreased after implantation of the device according to Method 2. FIG. 17C shows an increase in EF % from 20% to 22.9% between screening and three months post-implantation for Patient 10. This suggests a slight improvement in cardiac function of Patient 10 after implantation of the device according to Method 2. A comparison of the bar graphs of FIGS. 16A-C (Method 1), to the bar graphs of FIGS. 17A-C (Method 2) demonstrate improved efficacy of Method 1 compared to Method 2. The average EF % for multiple patients treated according to Method 1 increased by 10% from screening to three months, whereas EF % for two of the three patients (Patients 8 and 9) treated according to Method 2 decreased, and EF % for Patient 10 treated according to Method 2 increased by 2.9%. Thus, the data suggests that devices implanted according to Method 1 lead to improved cardiac function as indicated by increased EF % values when compared to Method 2.

FIGS. 18A-C depict a series of bar graphs showing average left ventricle end systolic volume (LVESV) over multiple patients who have been treated according to Method 1. LVESV was measured at one month, three months, and six months post-implantation and compared to LVESV at screening (i.e., prior to implantable device implantation). LVESV is a measure of the amount of blood left in the heart after the heart has fully contracted. That is, LVESV is a measurement of the lowest volume of blood in the heart during the cardiac cycle. In a functional heart, a low end systolic volume indicates that the heart is effectively pumping blood out of the ventricle. Therefore, the notable decrease in LVESV at one, three, and six months compared to screening shown in FIGS. 18A-18C indicate improved cardiac function of patients that were treated with devices implanted according to Method 1. FIG. 18A depicts average LVESV for 10 patients at screening versus average LVESV at one month post-implantation. LVESV decreased by 23% between screening and one month post-implantation. FIG. 18B depicts average LVESV for 9 patients at screening versus average LVESV at three months post-implantation. LVESV decreased by 21% from screening to three months post-implantation. FIG. 18C depicts average LVESV for 7 patients at screening versus LVESV at six months post-implantation. LVESV decreased by 31% from screening to six months post-implantation—a reduction in LVESV of 41 mL. Experiments using other implantable devices and methods typically show LVESV reductions on the order of 3 mL or less at 6 months post-implantation. FIG. 18D depicts a table of EF % and LVESV data for patients that have been treated with other mitral valve repair or replacement devices and summarizes data from two studies: (1) Acker M A, et al. “Mitral-Valve Repair versus Replacement for Severe Ischemic Mitral Regurgitation” N Engl J Med 2014; 370:23-32; and (2) Asch F M. “COAPT: Cardiovascular Outcomes Assessment of the MitraClip Percutaneous Therapy for Heart Failure Patients With Functional Mitral Regurgitation” presented at ACC 2019. MV repair and MV replacement therapies (Acker et al.) demonstrated single-digit reductions in the ESV index of about −7 and −5 respectively. MitraClip “Mitra-FR” and “COAPT” therapies demonstrated increases in the ESV index of about +1 and about +15, respectively. In comparison, as shown in FIGS. 18A-18C, patients treated using Method 1 demonstrated double-digit LVESV reductions. This is a surprisingly large improvement in cardiac function as compared to the treatment devices and methods of FIG. 18D.

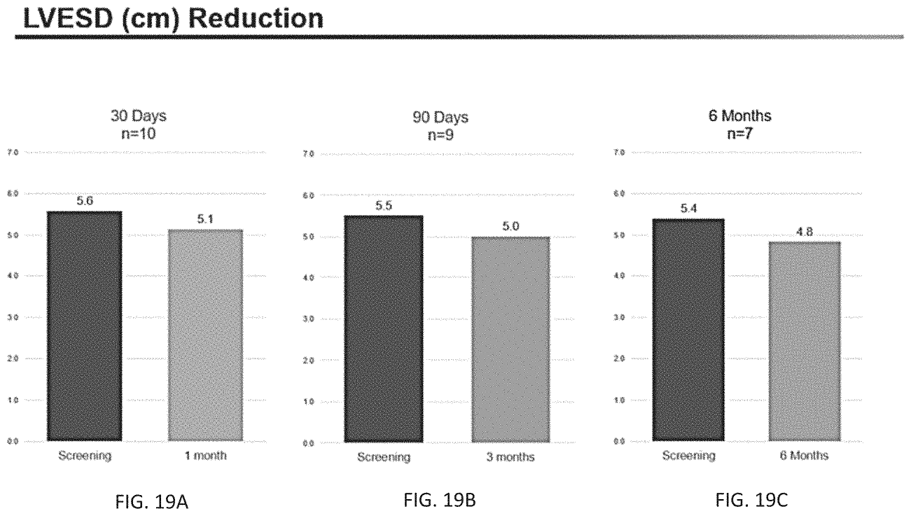

FIGS. 19A-C depict a series bar graphs showing average left ventricle end systolic dimension (LVESD) over multiple patients who have been treated according to Method 1. LVESD was measured at one month, three months, and six months post-implantation and compared to LVESD at screening. LVESD is a measurement of the diameter of the ventricle at the end of systole, when the heart is in its most contracted state. If the diameter of the ventricle does not decrease sufficiently during contraction, the heart will pump less blood than a fully functional heart. Therefore, the reduction in LVESD at one, two, and three months post-implantation as compared to LVESD at the time of screening shows in FIGS. 19A-19C indicates improved cardiac function of patients that were treated with devices implanted according to Method 1. FIG. 19A depicts average LVESD at screening and at one month post-implantation, demonstrating an average decrease of 0.5 cm. FIG. 19B depicts average LVESD at screening and at three months post-implantation, demonstrating an average decrease of 0.5 cm. FIG. 19C depicts average LVESD at screening and at six months post-implantation, demonstrating an average decrease of 0.6 cm.

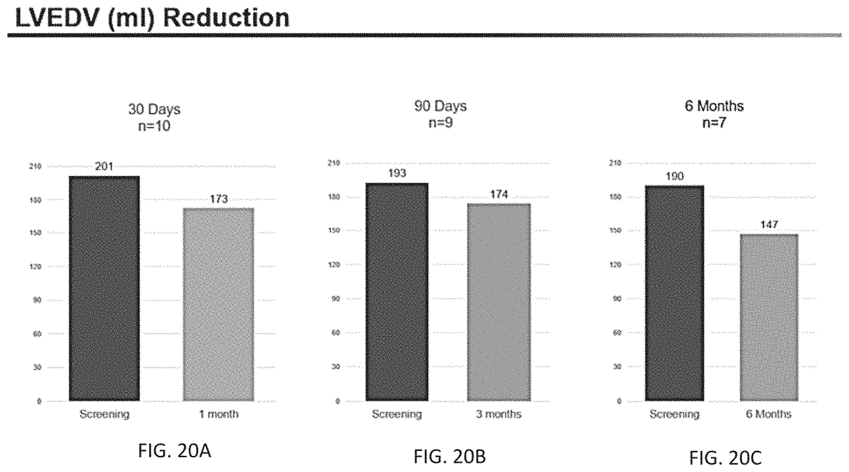

FIG. 20A-C depict a series of bar graphs showing average left ventricle end diastolic volume (LVEDV) over multiple patients who have been treated according to Method 1. LVEDV was measured at one month, three months, and six months post-implantation and compared to LVEDV at screening. LVEDV is the volume of blood in the ventricle at the end of diastole, when the heart is fully expanded and contains the greatest volume of blood in a heart cycle. In patients with mitral valve regurgitation, blood flows back into the atrium as the left ventricle contracts. This causes the left atrium to become engorged, which increases atrial pressure. Therefore, during left ventricle filling, the higher pressure and volume of the left atrium creates an increase in LVEDV. Thus, the reduction in LVEDV at one month, three months, and six months post-implantation as compared to LVEDV at the time of screening indicates that the severity of mitral valve regurgitation has been reduced, which indicates improved cardiac function of patients that were treated with devices implanted according to Method 1. FIG. 20A depicts average LVEDV at screening and at one month post-implantation, demonstrating an average decrease of 28 mL over 10 patients. FIG. 20B depicts average LVEDV at screening and at three months post-implantation, demonstrating an average decrease of 19 mL for 9 patients. FIG. 20C depicts average LVEDV at screening and at six months post-implantation, demonstrating an average decrease of 43 mL for 7 patients.

FIGS. 21A-21C depict a series of plots showing left ventricle end systolic volume and left ventricle end diastolic volume at screening, one month post-implantation, and three months post-implantation for individual patients treated according to Method 2. Each plot contains LVESV and LVEDV values for a single patient measured over time. FIG. 21A depicts LVESV and LVEDV values for Patient 11, FIG. 21B depicts LVESV and LVEDV values for Patient 12, and FIG. 21C depicts LVESV and LVEDV values for Patient 13. As discussed above, a lower LVESV indicates improved cardiac function because it demonstrates that the heart is able to effectively empty the ventricle during systole. The LVESV line in FIG. 21A shows an increase in LVESV for Patient 11 from 76 mL to 80 mL from screening to three months post-implantation. This increase in LVESV over time indicates decreased cardiac function. The LVESV line in FIG. 21B shows an increase in LVESV for Patient 12 from 53 mL to 92 mL from screening to three months post-implantation. This increase over time indicates decreased cardiac function. The LVESV line in FIG. 21C shows a decrease in LVESV for Patient 13 from 140 mL to 138 mL from screening to three months post-implantation. A comparison between FIGS. 18A-18C and FIGS. 21A-21C demonstrates the effectiveness of Method 1 compared to Method 2. For patients treated according to Method 1, FIGS. 18A-C demonstrate a decrease in LVESV over time (e.g. by 21% from screening to three months post-implantation). By contrast, FIGS. 21A-21C demonstrate an increase in LVESV over time for Patients 11 and 12, and a slight decrease (<2% from screening to three months) for Patient 13. Thus, the data suggests that implantation according to Method 1 leads to improved cardiac function as indicated by decreased LVESV when compared to Method 2.

FIGS. 21A-21C also show LVEDV measurements for Patients 11, 12, and 13 at screening, one month post-implantation, and three months post-implantation. As discussed above, a decrease in LVEDV indicates a reduction in the severity of mitral valve regurgitation, which suggests an improvement in cardiac function. The LVEDV line in FIG. 21A shows a decrease in LVEDV over time for Patient 11, from 124 mL to 117 mL between screening and three months. This indicates a reduction in the severity of mitral valve regurgitation over time. The LVEDV line in FIG. 21B shows an increase in LVEDV over time for Patient 12, from 114 mL to 186 mL between screening and three months. This indicates an increase in the severity of mitral valve regurgitation over time. The LVEDV line in FIG. 21C shows in increase in LVEDV over time for Patient F, from 175 mL to 179 mL between screening and three months. This indicates an increase in the severity of mitral valve regurgitation over time. A comparison between FIGS. 20A-C, and the LVEDV lines in FIGS. 21A-21C demonstrates the effectiveness of Method 1 compared to Method 2 in decreasing left ventricle end diastolic volume. For patients treated according to Method 1, FIG. 20B demonstrates a decrease in average LVEDV for multiple patients from 193 mL to 174 mL (a 19 mL decrease) between screening and three months. For patients treated according to Method 2, FIGS. 21A-21C demonstrate an increase in LVEDV for Patients 12 and 13, and a decrease in LVEDV of 7 mL for Patient 11. Thus, the data suggests that implantation according to Method 1 leads to improved cardiac function as indicated by decreased LVEDV when compared to Method 2.

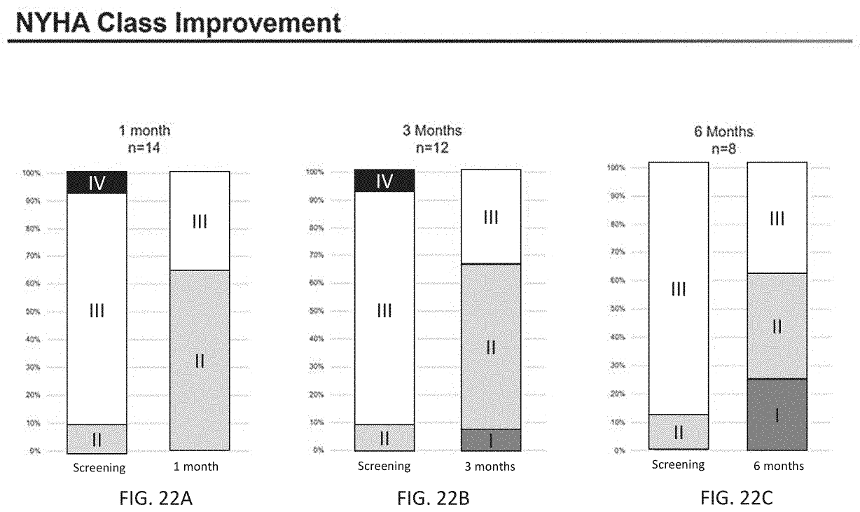

FIGS. 22A-22C depict a series of bar graphs showing the New York Heart Association (NYHA) Functional Classification of multiple patients treated according to Method 1. The NYHA Functional Classification (“NYHA Class”) is a classification of the severity of a patient's heart failure. The NYHA has four classes based on how much a patient is limited during physical activity, with Class IV being the most severe form of heart failure, and Class I being the least. Thus, a lower NYHA class indicates a decrease in the severity of heart failure, and suggests improved cardiac function. The bar graphs in FIG. 22A show the percentage of patients in each NHYA Class at screening and at one month after treatment according to Method 1. From screening to one month post-implantation, the percentage of patients in Class IV (the most severe heart failure classification) decreased to zero, the percentage of patients in Class III decreased from approximately 85% to approximately 35%, and the percentage of patients in Class II increased from approximately 8% to approximately 65%. This demonstrates that the overall level of severity of heart failure of patients treated according to Method 1 decreased from the time of screening to one month post-implantation. FIG. 22B shows the percentage of patients in each NYHA class at screening and at three months after treatment according to Method 1. Three months after implantation, the percentage of patients in Class IV remained the same, the percentage of patients in Class III decreased from approximately 85% to approximately 35%, the percentage of patients in Class II increased from approximately 8% to approximately 60%, and the percentage of patients in Class I increased from none at screening (i.e. 0) to approximately 5%. This demonstrates that the overall level of severity of heart failure of patients treated according to Method 1 decreased from screening to three months post-implantation, where some patients who had higher degrees of heart failure improved to a lower degree of heart failure (e.g., moving from NYHA Class III to Class II, and Class II to Class I). FIG. 22C shows the percentage of patients in each NYHA class at screening and at six months after treatment According to Method 1. Six months after implantation, none of the patients were in Class IV, the percentage of patients in Class III decreased from approximately 90% to approximately 40%, the percentage of patients in Class II increased from approximately 10% to approximately 35%, and the percentage of patients in Class I increased from none at screening to approximately 25%. This demonstrates that the overall level of severity of heart failure of patients treated according to Method 1 decreased from screening to six months post-implantation, where some patients who had higher degrees of heart failure improved to a lower degree of heart failure. For example, patients who were in the Class IV group one month and three months after implantation experienced improvements in cardiac function such that their heart failure severity was downgraded from Class IV.

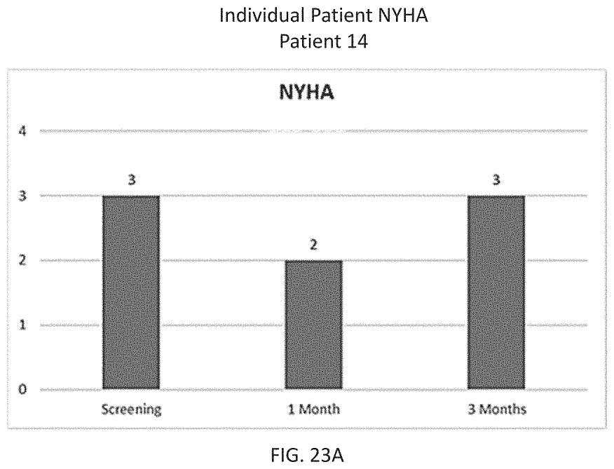

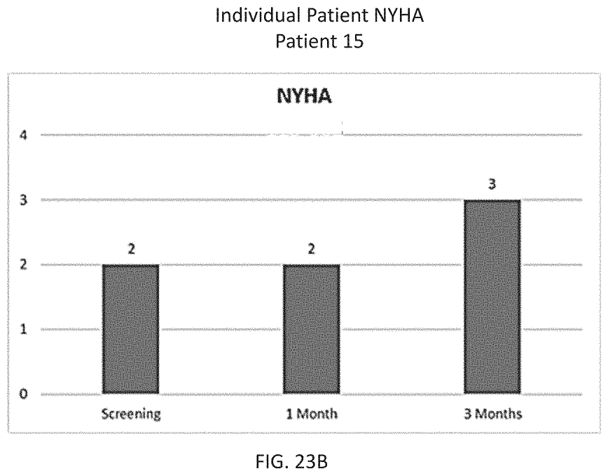

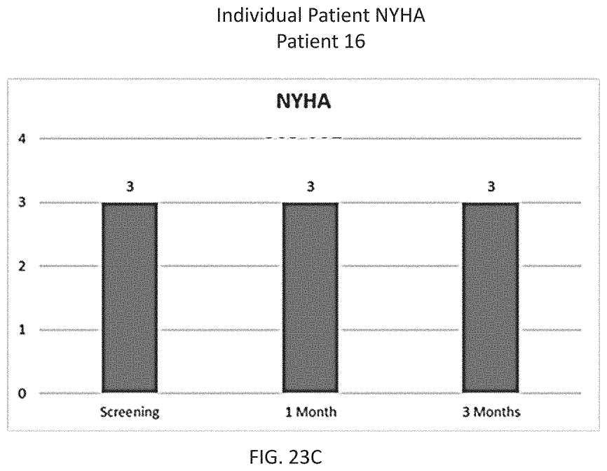

FIGS. 23A-23C depict a series of bar graphs showing the NYHA Class for individual patients screening, at one month post-implantation, and at three months post-implantation who have been treated according to Method 2. The bar graphs in FIG. 23A show the NYHA Class for Patient 14 at screening, one month, and three months post-implantation. The NYHA Class for Patient 14 did not change from screening to three months, indicating that the severity of this patient's heart failure remained unchanged. FIG. 23B shows the NYHA Class for Patient 15 at screening, one month, and three months post-implantation. The NYHA Class for Patient 15 increased from screening to three months, indicating that the severity of this patient's heart failure increased. FIG. 23C shows the NYHA Class for Patient 16 at screening, one month, and three months post-implantation. The NYHA Class for Patient 16 did not change at each time point, indicating that the severity of this patient's heart failure remained constant at Class III. When compared to FIGS. 22A and 22B, FIGS. 23A-23C demonstrate that Method 2 is not as effective as Method 1 in decreasing the severity of patient's heart failure. For patients treated according to Method 1, FIGS. 22A and 22B depict an overall decrease in severity of heart failure over time, as indicated by the reduction in patients exhibiting Class III and IV symptoms from screening to one month post-implantation and from screening to three months post-implantation. By contrast, the severity of the heart failure in patients treated according to Method 2 generally increased or stayed constant over time, as depicted in FIGS. 23A-23C. Thus, the data suggests that implantation according to Method 1 leads to improved cardiac function as indicated by decreased NYHA Class when compared to Method 2.

Taken together, the experimental results and data described above indicate that implanting a cinchable device according to Method 1 leads to an unexpected and surprising synergy that promotes therapeutic cardiac remodeling and improves cardiac function. The surprising and unexpected improvement in cardiac function are evidenced by a plurality of cardiac function metrics, including, but not limited to EF %, LVESV, LVESD, LVEDV, NYHA, etc. In particular, the improvement in cardiac function is evident when comparing EF %, LVESV, LVEDV, and NYHA data collected for the methods described herein (Method 1) to alternative methods (Methods 2 and 3).

1. A method for reshaping a heart ventricle comprising:
securing a device into ventricular wall tissue approximately 10-20 mm below a mitral valve plane, wherein the device comprises a plurality of anchors coupled to a tether;
cinching the device from an uncinched configuration to a cinched configuration by tensioning the tether until a circumferential portion of the ventricle at a location of the device is reduced by approximately 30%; and
locking the device in the cinched configuration.

2. The method of claim 1, wherein the ventricular wall tissue is located between the mitral valve plane and a papillary muscle insertion location.

3. The method of claim 1, wherein securing the device into the ventricular wall tissue comprises implanting the plurality of anchors across approximately 220-230 degrees of a circumference of the ventricle.

4. The method of claim 1, wherein locking the device in the cinched configuration comprises securing a lock member at a terminal end of the device.

5. The method of claim 1, wherein locking the device in the cinched configuration further comprises introducing a pre-selected amount of slack to the tether.

6. The method of claim 5, wherein the plurality of anchors comprises a first anchor and a terminal anchor, and introducing a pre-selected amount of slack to the tether comprises securing a lock member on the tether at a pre-selected distance from the terminal anchor when the device is in the cinched configuration.

7. The method of claim 1, wherein the device extends around a circumference of the ventricle between a junction of a septum and a ventricular free wall adjacent the mitral valve P3 leaflet, and a ventricular outflow tract.

8. The method of claim 1, wherein when the device is in the uncinched configuration it has an uncinched length, and a ratio R between the uncinched length and an inner diameter of a ventricle at end-diastole has a magnitude of at about 2 or more.

9. The method of claim 1, wherein securing the device into ventricular wall tissue comprises securing a total of 11-16 anchors along ventricular wall tissue.

10. The method of claim 1, wherein securing the device into ventricular wall tissue comprises deploying each of the anchors into the ventricle wall sequentially.

11. The method of claim 1, wherein securing the device into ventricular wall tissue comprises deploying the plurality of anchors into the ventricle wall simultaneously.

12. The method of claim 1, wherein the device further comprises a plurality of force distributing members, wherein each force distributing member is coupled to the tether between two anchors.

13. The method of claim 1, wherein securing the device into ventricular wall tissue further comprises positioning a multi-window catheter in the ventricle approximately 10-20 mm below the mitral valve plane.

14. The method of claim 13, wherein the multi-window catheter comprises a reinforced distal end comprising a pre-defined curvature that approximates a curvature at a widest circumference of the ventricle.

15. The method of claim 1, wherein the mitral valve plane comprises a plane of a mitral valve annulus.

16. A method for reshaping a heart ventricle comprising:
implanting a device into ventricular wall tissue approximately 10-20 mm below a mitral valve plane; and
cinching the device from an uncinched configuration to a cinched configuration such that a circumferential portion of the ventricle at a location of the device is reduced by approximately 30%.

17. The method of claim 16, further comprising securing the device in the cinched configuration.

18. The method of claim 16, wherein when the device is in the uncinched configuration it has an uncinched length, and a ratio R between the uncinched length and an inner diameter of a ventricle at end-diastole has a magnitude of at least 2.

19. The method of claim 16, further comprising implanting the device in a plane that is substantially parallel to the mitral valve plane.

20. The method of claim 16, wherein implanting the device comprises attaching a portion of the device into the ventricular wall tissue.

21. The method of claim 16 wherein the ventricular wall tissue is located between a mitral valve plane and a papillary muscle insertion location.

22. The method of claim 16, wherein cinching the device from an uncinched configuration to a cinched configuration comprises applying tension to a portion of the device.

23. The method of claim 16, wherein the device comprises a shape-memory material and wherein implanting the device comprises restraining the device in an uncinched configuration, and wherein cinching the device comprises unrestraining the device such that transitions to the cinched configuration.

24. The method of claim 16, wherein implanting the device comprises securing the device to the ventricular wall tissue across approximately 220-230 degrees of a circumference of the ventricle.

25. The method of claim 16, wherein the device extends around a circumference of the ventricle between a junction of a septum and a ventricular free wall adjacent the mitral valve P3 leaflet, and a ventricular outflow tract.
